(19) United States
(12) Patent Application Publication
(10) Pub. No.: US 2006/0069101 A1
(43) Pub. Date: Mar. 30, 2006

(54) Prodrugs of protein tyrosine kinase inhibitors

(76) Inventors: Kyoung S. Kim, North Brunswick, NJ (US); Francis Y. Lee, Yardley, PA (US); Louis J. Lombardo, Belle Mead, NJ (US); Feng Roger Luo, Plainsboro, NJ (US); John Wityak, Carlsbad, CA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/235,507

(22) Filed: Sep. 26, 2005

Related U.S. Application Data
(60) Provisional application No. 60/613,540, filed on Sep. 27, 2004.

(51) Int. Cl. A61K 31/506; C07D 417/14

(52) U.S. Cl. 514/252.19; 544/295

(57) ABSTRACT

Novel compounds and salts thereof, pharmaceutical compositions containing such compounds, and methods of using such compounds in the treatment of protein tyrosine kinase-associated disorders such as oncologic and immunologic disorders.

### CROSS-REFERENCE TO RELATED APPLICATIONS

The present application claims the priority benefit of U.S. Provisional Application No. 60/613,540 filed Sep. 27, 2004, the entire disclosure of which is herein incorporated by reference.

### FIELD OF THE INVENTION

The present invention relates generally to prodrugs of aminothiazole piperazine analogues and salts thereof, to methods of using such compounds in treating protein tyrosine kinase-associated disorders such as oncologic and immunologic disorders, and to pharmaceutical compositions containing such compounds.

### BACKGROUND OF THE INVENTION

The compound of Formula A is a known protein tyrosine kinase inhibitors and is disclosed in U.S. Ser. No. 09/548,929, filed Apr. 13, 2000, now U.S. Pat. No. 6,596,746, the contents of which are hereby incorporated by reference.

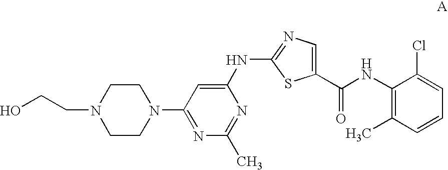

Since improved pharmacokinetic properties are desired, it is desirable to discover new compounds that provide better pharmacokinetic properties.

### SUMMARY OF THE INVENTION

The present invention provides compounds of the following formula (Ia), (Ib), and/or (Ic) and salts thereof, for use as protein tyrosine kinase inhibitors: 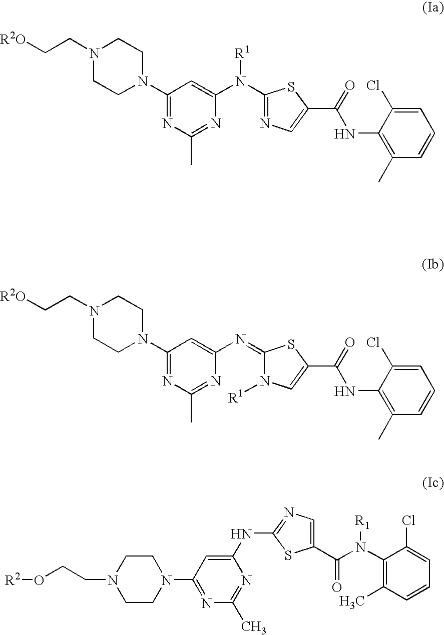
or stereoisomer or pharmaceutically acceptable salt form thererof.

Accordingly, the present invention provides novel inhibitors of protein tyrosine kinase or pharmaceutically acceptable salts or prodrugs thereof.

The present invention provides pharmaceutical compositions comprising a pharmaceutically acceptable carrier and a therapeutically effective amount of at least one of the compounds of the present invention or a pharmaceutically acceptable salt or prodrug form thereof.

The present invention provides pharmaceutical compositions comprising a pharmaceutically acceptable carrier and an amount of a compound of formula (Ia), (Ib), and/or (Ic) to provide a therapeutically effective amount of at least one of the compounds of the present invention or a pharmaceutically acceptable salt or prodrug form thereof.

The present invention provides novel compounds for use in therapy.

The present invention provides the use of novel compounds for the manufacture of a medicament for the treatment of oncological or immunological diseases.

The present invention is directed to a compound of formula (Ia), (Ib), and/or (Ic) or a salt thereof: 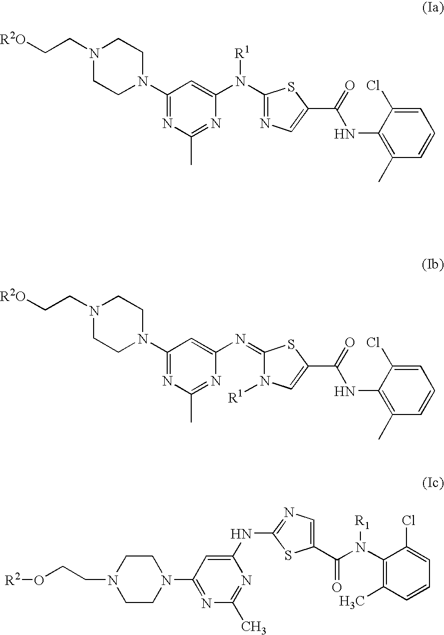
or stereoisomer or pharmaceutically acceptable salt form thererof, wherein
R¹ is selected from H, —P(O)(ONa)₂, —C(R^{a})₂—R³, —C(O)—R, —C(O)O—R;
R² is selected from H, —P(O)(ONa)₂, —C(R^{a})₂—R³, —C(O)—R, —C(O)O—R, —C(O)N(R^{a})₂;
with the provisio that R¹ and R² do not both equal H;
R is independently selected from alkyl, haloalkyl, alkenyl, alkynyl, aryl, heteroaryl, cycloalkyl, carbocyclyl, heterocyclyl, each optionally substituted with one or more Z₁;
R³ is selected from NHC(O)R^{a}, OC(O)R^{a}, —OP(O)(ONa)₂, NHC(O)OR, OC(O)OR, OC(O)NHR, 5-methyl-2-oxo-[1,3]dioxol-4-yl, and alkyl 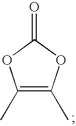
Z¹ is selected from alkyl, haloalkyl, alkenyl, alkynyl, aryl, heteroaryl, cycloalkyl, carbocyclyl, heterocyclyl, NR^{a}R^{a}, OR^{a}, NC(O)R^{a}, OC(O)R^{a}; and
R^{a} is independently selected from hydrogen, alkyl, haloalkyl, alkenyl, alkynyl, aryl, heteroaryl, cycloalkyl, carbocyclyl, heterocyclyl.

In another embodiment, the present invention is directed to a compound of formula (Ia) wherein
R¹ is H; and
R² is selected from —P(O)(ONa)₂, —C(R^{a})₂—R³, —C(O)—R, —C(O)O—R, —C(O)N(R^{a})₂.

In another embodiment, the present invention is directed to a compound of formula (Ia) wherein
R² is selected from —P(O)(ONa)₂, —C(O)—R, —C(O)O—R; and
R is independently selected from alkyl, haloalkyl, aryl, heteroaryl, cycloalkyl, heterocyclyl, each optionally substituted with one or more Z₁.

In another embodiment, the present invention is directed to a compound of formula (Ia) wherein
R is independently selected from alkyl, haloalkyl, each optionally substituted with one or more Z₁;
Z₁ is independently selected from alkyl and NR^{a}R^{a}; and
R^{a} is independently selected from hydrogen, alkyl.

In another embodiment, the present invention is directed to a compound of formula (Ia), (Ib), and/or (Ic) wherein
R is independently selected from methyl, ethyl, propyl, i-propyl, butyl, s-butyl, i-butyl, t-butyl, pentyl, hexyl, each optionally substituted with one or more Z₁.

In another embodiment, the present invention is directed to a compound of formula (Ia), (Ib), and/or (Ic), wherein
R¹ is selected from —P(O)(ONa)₂, —C(R^{a})₂—R³, —C(O)—R, —C(O)O—R;
R² is selected from H.

In another embodiment, the present invention is directed to a compound of formula (Ia), (Ib), and/or (Ic), wherein
R¹ is selected from —P(O)(ONa)₂, —C(R^{a})₂—R³, —C(O)—R, —C(O)O—R;
R is independently selected from alkyl, haloalkyl, aryl, heteroaryl, cycloalkyl, heterocyclyl, each optionally substituted with one or more Z₁;

R³ is selected from NHC(O)R^{a}, OC(O)R^{a}, —OP(O)(ONa)₂, NHC(O)OR, OC(O)OR, OC(O)NHR, and alkyl

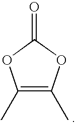

In another embodiment, the present invention is directed to a compound of formula (Ia), (Ib), and/or (Ic), wherein
R is independently selected from alkyl, haloalkyl, each optionally substituted with one or more Z₁;
Z₁ is independently selected from alkyl and NR^{a}R^{a}; and
R^{a} is independently selected from hydrogen, alkyl.

In another embodiment, the present invention is directed to a compound of formula (Ia), (Ib), and/or (Ic), wherein
R¹ is selected from —C(R^{a})₂—R³, —C(O)O—R;
R is independently selected from methyl, ethyl, propyl, i-propyl, butyl, s-butyl, i-butyl, t-butyl, pentyl, hexyl, each optionally substituted with one or more Z₁;
R³ is OC(O)R^{a} and alkyl

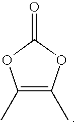

In another embodiment, the present invention is directed to a compound of formula (Ia), (Ib), and/or (Ic), wherein
R¹ is selected from —CH₂—R³, —C(O)O—R;

In another embodiment, the present invention is directed to a compound of formula (Ia).

In another embodiment, the present invention is directed to a compound of formula (Ib).

In another embodiment, the present invention is directed to a compound of formula (Ic).

In another embodiment, the present invention is directed to a pharmaceutical composition, comprising a pharmaceutically acceptable carrier and a therapeutically effective amount of a compound of Formula (Ia), (Ib), and/or (Ic).

In another embodiment, the present invention is directed to a method for the treatment of a protein tyrosine kinase-associated disorder, comprising the step of administering to a subject in need thereof an amount effective therefor of at least one compound Formula (Ia), (Ib), and/or (Ic) or a salt thereof

In another embodiment, the present invention is directed to a method, wherein said protein tyrosine kinase-associated disorder is selected from transplant rejection, rheumatoid arthritis, multiple sclerosis, lupus, graft vs. host disease, a T-cell mediated hypersensitivity disease, psoriasis, Hashimoto's thyroiditis, Guillain-Barre syndrome, cancer, contact dermatitis, an allergic disease, asthma ischemic or reperfusion injury, atopic dermatitis, allergic rhinitis, chronic obstructive pulmonary disease, diabetic retinopathy.

In another embodiment, the present invention is directed to a method, wherein said protein tyrosine-kinase-associated disorder is cancer.

In another embodiment, the present invention is directed to a method for the treatment of a protein tyrosine kinase-associated disorder, wherein the cancer is chronic myelogenous leukemia (CML), gastrointestinal stromal tumor (GIST), acute lymphoblastic leukemia (ALL), Philadelphia positive ALL, small cell lung cancer (SCLC), non-small cell lung cancer (NSCLC), ovarian cancer, melanoma, mastocytosis, germ cell tumors, acute myelogenous leukemia (AML), pediatric sarcomas, breast cancer, colorectal cancer, pancreatic cancer, or prostate cancer.

In another embodiment, the present invention is directed to a method, wherein said compound of the formula (Ia), (Ib), and/or (Ic) or salt thereof is administered with one or more of: another PTK inhibitor; cyclosporin A; CTLA4-Ig; antibodies selected from anti-ICAM-3, anti-IL-2 receptor (Anti-Tac), anti-CD45RB, anti-CD2, anti-CD3 (OKT-3), anti-CD4, anti-CD80, anti-CD86, and monoclonal antibody OKT3; agents blocking the interaction between CD40 and gp39; fusion proteins constructed from CD40 and gp39; inhibitors of NF-kappa B function; non-steroidal antiinflammatory drugs (NSAIDs); steroids; gold compounds; antiproliferative agents; FK506 (tacrolimus, Prograf); mycophenolate mofetil; cytotoxic drugs; TNF-α inhibitors; anti-TNF antibodies or soluble TNF receptor; rapamycin (sirolimus or Rapamune); leflunimide; cyclooxygenase-2 inhibitors; paclitaxel, cisplatin, carboplatin, doxorubicin, carminomycin, daunorubicin, aminopterin, methotrexate, methopterin, mitomycin C, ecteinascidin 743, porfiromycin, 5-fluorouracil, 6-mercaptopurine, gemcitabine, cytosine arabinoside, podophyllotoxin, etoposide, etoposide phosphate, teniposide, melphalan, vinblastine, vincristine, leurosidine, epothilone, vindesine, leurosine, or derivatives thereof.

In another embodiment, the present invention is directed to a method of, wherein said compound of the formula (Ia), (Ib), and/or (Ic) or salt thereof is administered with one or more of: linomide, inhibitors of integrin αvβ3 function, angiostatin, razoxane, tamoxifen, toremifene, raloxifene, droloxifene, iodoxifene, megestrol acetate, anastrozole, letrozole, borazole, exemestane, flutamide, nilutamide, bicalutamide, cyproterone acetate, gosereline acetate, leuprolide, finasteride, herceptin, metalloproteinase inhibitors, inhibitors of urokinase plasminogen activator receptor function, growth factor antibodies, growth factor receptor antibodies, bevacizumab, cetuximab, tyrosine kinase inhibitors, serine/threonine kinase inhibitors, methotrexate, 5-fluorouracil, purine, adenosine analogues, cytosine arabinoside, doxorubicin, daunomycin, epirubicin, idarubicin, mitomycin-C, dactinomycin, mithramycin, cisplatin, carboplatin, nitrogen mustard, melphalan, chlorambucil, busulphan, cyclophosphamide, ifosfamide, nitrosoureas, thiotepa, vincristine, paclitaxel, docetaxel, epothilone analogs, discodermolide analogs, eleutherobin analogs, etoposide, teniposide, amsacrine, topotecan, irinotecan, flavopyridols, proteasome inhibitors including bortezomib and biological response modifiers.

The invention also provides a pharmaceutical composition comprising a compound of formula (Ia), (Ib), and/or (Ic) and a pharmaceutically acceptable carrier.

The invention also provides a pharmaceutical composition comprising a compound of formula (Ia), (Ib), and/or (Ic) in combination with pharmaceutically acceptable carrier and an anti-cancer or cytotoxic agent. In a preferred embodiment said anti-cancer or cytotoxic agent is selected from the group consisting of linomide; inhibitors of integrin αvβ3 function; angiostatin, razoxane, tamoxifen, toremifene; raloxifene, droloxifene, iodoxifene, megestrol acetate, anastrozole, letrozole, borazole, exemestane, flutamide, nilutamide, bicalutamide, cyproterone acetate, gosereline acetate, leuprolide, finasteride; metalloproteinase inhibitors; inhibitors of urokinase plasminogen activator receptor function; growth factor antibodies; growth factor receptor antibodies such as Avastin® (bevacizumab) and Erbitux® (cetuximab); tyrosine kinase inhibitors such as Gleevec® (imatinib mesylate) and AMN-107; serine/threonine kinase inhibitors; methotrexate, 5-fluorouracil, purine, adenosine analogues, cytosine arabinoside, doxorubicin, daunomycin, epirubicin, idarubicin, mitomycin-C, dactinomycin, mithramycin, cisplatin, carboplatin, nitrogen mustard, melphalan, chlorambucil, busulphan, cyclophosphamide, ifosfamide, nitrosoureas, thiotepa, vincristine, Taxol® (paclitaxel), Taxotere® (docetaxel), epothilone analogs, discodermolide analogs, eleutherobin analogs, etoposide, teniposide, amsacrine, topotecan, flavopyridols; biological response modifiers and proteasome inhibitors such as Velcade® (bortezomib).

The invention also provides a method of inhibiting protein kinase activity of growth factor receptors which comprises administering to a mammalian species in need thereof, a therapeutically effective protein kinase inhibiting amount of a compound of formula (Ia), (Ib), and/or (Ic).

Finally, there is disclosed a method for treating a proliferative disease, comprising administering to a mammalian species in need thereof, a therapeutically effective amount of a compound of formula (Ia), (Ib), and/or (Ic). In another embodiment the proliferative disease is cancer.

The invention may be embodied in other specific forms without departing from the spirit or essential attributes thereof. This invention also encompasses all combinations of aspects of the invention noted herein. It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments.

The following are definitions of terms used in this specification. The initial definition provided for a group or term herein applies to that group or term throughout the present specification, individually or as part of another group, unless otherwise indicated.

The terms “alk” or “alkyl” refer to straight or branched chain hydrocarbon groups having 1 to 12 carbon atoms, preferably 1 to 8 carbon atoms. The expression “lower alkyl” refers to alkyl groups of 1 to 4 carbon atoms. The term “haloalkyl” is intended to include both branched and straight-chain saturated aliphatic hydrocarbon groups, for example CF₃, having the specified number of carbon atoms, substituted with 1 or more halogen (for example —CᵥF_{w} where v=1 to 3 and w=1 to (2v+1)).

The term “alkenyl” refers to straight or branched chain hydrocarbon groups of 2 to 10, preferably 2 to 4, carbon atoms having at least one double bond. Where an alkenyl group is bonded to a nitrogen atom, it is preferred that such group not be bonded directly through a carbon bearing a double bond.

The term “alkynyl” refers to straight or branched chain hydrocarbon groups of 2 to 10, preferably 2 to 4, carbon atoms having at least one triple bond. Where an alkynyl group is bonded to a nitrogen atom, it is preferred that such group not be bonded directly through a carbon bearing a triple bond.

The term “alkylene” refers to a straight chain bridge of 1 to 5 carbon atoms connected by single bonds (e.g., —(CH₂)ₓ— wherein x is 1 to 5), which may be substituted with 1 to 3 lower alkyl groups.

The term “alkenylene” refers to a straight chain bridge of 2 to 5 carbon atoms having one or two double bonds that is connected by single bonds and may be substituted with 1 to 3 lower alkyl groups. Exemplary alkenylene groups are —CH═CH—CH═CH—, —CH₂—CH═CH—, —CH₂—CH═CH—CH₂—, —C(CH₃)₂CH═CH— and —CH(C₂H₅)—CH═CH—.

The term “alkynylene” refers to a straight chain bridge of 2 to 5 carbon atoms that has a triple bond therein, is connected by single bonds, and may be substituted with 1 to 3 lower alkyl groups. Exemplary alkynylene groups are —C≡C—, —CH₂—C≡C—, —CH(CH₃)—C≡C— and —C≡C—CH(C₂H₅)CH₂—.

The terms “ar” or “aryl” refer to aromatic cyclic groups (for example 6 membered monocyclic, 10 membered bicyclic or 14 membered tricyclic ring systems) which contain 6 to 14 carbon atoms. Exemplary aryl groups include phenyl, naphthyl, biphenyl and anthracene.

The terms “cycloalkyl” and “cycloalkenyl” refer to cyclic hydrocarbon groups of 3 to 12 carbon atoms.

The terms “carbocycle”, “carbocyclyl”, and “carbocyclic” refer to cycloalkyl and/or aryl.

The terms “halogen” and “halo” refer to fluorine, chlorine, bromine and iodine.

The term “unsaturated ring” includes partially unsaturated and aromatic rings.

The terms “heterocycle”, “heterocyclic” “heterocyclyl” or “heterocyclo” refer to fully saturated or unsaturated, including non-aromatic (i.e. “heterocycloalkyl) and aromatic (i.e. “heteroaryl”) cyclic groups, for example, 4 to 7 membered monocyclic, 7 to 11 membered bicyclic, or 10 to 15 membered tricyclic ring systems, which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, triazinyl, and the like.

Exemplary bicyclic heterocyclic groups include indolyl, benzothiazolyl, benzoxazolyl, benzodioxolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), tetrahydroquinolinyl and the like.

Exemplary tricyclic heterocyclic groups include carbazolyl, benzidolyl, phenanthrolinyl, acridinyl, phenanthridinyl, xanthenyl and the like.

The term “heteroaryl” refers to aromatic heterocyclic groups.

Exemplary heteroaryl groups include pyrrolyl, pyrazolyl, imidazolyl, oxazolyl, isoxazolyl, thiazolyl, thiadiazolyl, isothiazolyl, furyl, thienyl, oxadiazolyl, pyridinyl, pyrazinyl, pyrimidinyl, pyridazinyl, triazolyl, triazinyl, and the like.

Compounds of the formula I may in some cases form salts which are also within the scope of this invention. Reference to a compound of the formula I herein is understood to include reference to salts thereof, unless otherwise indicated. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the R substituents comprise an acid moiety such as a carboxyl group). Also included herein are quaternary ammonium salts such as alkylammonium salts. Pharmaceutically acceptable (i.e., non-toxic, physiologically acceptable) salts are preferred, although other salts are useful, for example, in isolation or purification steps which may be employed during preparation. Salts of the compounds of the formula I may be formed, for example, by reacting a compound I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts (formed, for example, where the R substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. The basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All stereoisomers of the present compounds, such as those which may exist due to asymmetric carbons on the R substituents of the compound of the formula I, including enantiomeric and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

“Therapeutically effective amount” is intended to include an amount of a compound of the present invention alone or an amount of the combination of compounds claimed or an amount of a compound of the present invention in combination with other active ingredients effective to inhibit protein kinase activity or effective to treat or prevent oncological or immunological disorders. The term may alternatively be an amount of a compound of the present invention, which when administered as a prodrug is converted to an amount of compound A which is effective to inhibit protein kinase activity or effective to treat or prevent oncological or immunological disorders.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

Throughout the specification, groups and substituents thereof are chosen to provide stable moieties and compounds.

When any variable (e.g., R) occurs more than one time in any constituent or formula for a compound, its definition at each occurrence is independent of its definition at every other occurrence. Thus, for example, if a group is shown to be substituted with 0-2 R, then said group may optionally be substituted with up to two R groups and R at each occurrence is selected independently from the definition of R. Also, combinations of substituents and/or variables are permissible only if such combinations result in stable compounds.

The position of R¹ in the compounds of formula (Ia) and (Ib) are tautomers of each other. It is to be understood that some substituents may be more stable in the structure indicated by formula (Ia) while others may be more stable in the structure indicated by formula (Ib). Each of these regioisomers are intended to be covered by this invention.

### UTILITY

The compounds of formula (Ia), (Ib), and/or (Ic) are expected to possess protein tyrosine kinase inhibitory activity either by themselves and/or after administration and removal of the prodrug groups and are therefore useful as agents for the treatment, including prevention and therapy, of protein tyrosine kinase-associated disorders such as oncologic and immunologic disorders. Following administration, the prodrug group is expected to be removed by chemical or enzymatic processes thereby releasing the active hydroxy-agent.

The compounds of the present invention and/or the compound of formula A after administration of the compound of the present invention inhibit protein tyrosine kinases, especially Src-family kinases such as Lck, Fyn, Lyn, Src, Yes, Hck, Fgr and Blk, and are thus useful in the treatment, including prevention and therapy, of protein tyrosine kinase-associated disorders such as oncologic and immunologic disorders. The compounds inhibit also receptor tyrosine kinases including HER1 and HER2 and are therefore useful in the treatment of proliferative disorders such as psoriasis and cancer. The ability of these compounds to inhibit HER1 and other receptor kinases will also permit their use as anti-angiogenic agents to treat disorders such as cancer and diabetic retinopathy. “Protein tyrosine kinase-associated disorders” are those disorders which result from aberrant tyrosine kinase activity, and/or which are alleviated by the inhibition of one or more of these enzymes. For example, Lck inhibitors are of value in the treatment of a number of such disorders (for example, the treatment of autoimmune diseases), as Lck inhibition blocks T cell activation. The treatment of T cell mediated diseases, including inhibition of T cell activation and proliferation, is a particularly preferred embodiment of the present invention. Compounds which selectively block T cell activation and proliferation are preferred. Compounds of the present invention which block the activation of endothelial cell PTK by oxidative stress, thereby limiting surface expression of adhesion molecules that induce neutrophil binding, and which inhibit PTK necessary for neutrophil activation are useful, for example, in the treatment of ischemia and reperfusion injury.

The present invention thus provides methods for the treatment of protein tyrosine kinase-associated disorders, comprising the step of administering to a subject in need thereof at least one compound of the formula (Ia), (Ib), and/or (Ic) in an amount effective therefor. Other therapeutic agents such as those described below may be employed with the inventive compounds in the present methods. In the methods of the present invention, such other therapeutic agent(s) may be administered prior to, simultaneously with or following the administration of the compound(s) of the present invention.

The compounds of the present invention are useful for the treatment of cancers such as chronic myelogenous leukemia (CML), gastrointestinal stromal tumor (GIST), acute lymphoblastic leukemia (ALL), Philadelphia positive ALL, small cell lung cancer (SCLC), non-small cell lung cancer (NSCLC), ovarian cancer, melanoma, mastocytosis, germ cell tumors, acute myelogenous leukemia (AML), pediatric sarcomas, breast cancer, colorectal cancer, pancreatic cancer, prostate cancer and others known to be associated with protein tyrosine kinases such as, for example, SRC, BCR-ABL and c-KIT. The compounds of the present invention are also useful in the treatment of cancers that are sensitive to and resistant to chemotherapeutic agents that target BCR-ABL and c-KIT, such as, for example, Gleevec® (STI-571) and AMN-107.

Thus, the present invention provides methods for the treatment of a variety of cancers, including, but not limited to, the following:
carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma);
hematopoietic tumors of lymphoid lineage including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, histiocytic lymphoma, and Burketts lymphoma;
hematopoietic tumors of myeloid lineage including acute and chronic myelogenous leukemias, myelodysplastic syndrome, myeloid leukemia, and promyelocytic leukemia;
tumors of the central and peripheral nervous system including astrocytoma, neuroblastoma, glioma, and schwannomas;
tumors of mesenchymal origin including fibrosarcoma, rhabdomyoscarcoma, and osteosarcoma; and
other tumors including melanoma, xenoderma pigmentosum, keratoactanthoma, seminoma, thyroid follicular cancer, and teratocarcinoma.

The present invention provides methods for the treatment of a variety of non-cancerous proliferative diseases. The invention is used to treat GIST, Breast cancer, pancreatic cancer, colon cancer, NSCLC, CML, and ALL, sarcoma, and various pediatric cancers.

Use of the compounds of the present invention in treating protein tyrosine kinase-associated disorders is exemplified by, but is not limited to, treating a range of disorders such as: cancer, transplant (such as organ transplant, acute transplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; chronic obstructive pulmonary disease (COPD), such as emphysema; inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; cancers, including cancers where Lck or other Src-family kinases such as Src are activated or overexpressed, such as colon carcinoma and thymoma, and cancers where Src-family kinase activity facilitates tumor growth or survival; glomerulonephritis; serum sickness; uticaria; allergic diseases such as respiratory allergies (asthma, hayfever, allergic rhinitis) or skin allergies; scleracierma; mycosis fungoides; acute inflammatory responses (such as acute respiratory distress syndrome and ishchemia/reperfusion injury); dermatomyositis; alopecia areata; chronic actinic dermatitis; eczema; Behcet's disease; Pustulosis palmoplanteris; Pyoderma gangrenum; Sezary's syndrome; atopic dermatitis; systemic schlerosis; and morphea. The present invention also provides a method for treating the aforementioned disorders such as atopic dermatitis by administration of any compound capable of inhibiting protein tyrosine kinase.

Src-family kinases other than Lck, such as Hck and Fgr, are important in the Fc gamma receptor responses of monocytes and macrophages. Compounds of the present invention inhibit the Fc gamma dependent production of TNF alpha in the monocyte cell line THP-1 that does not express Lck. The ability to inhibit Fc gamma receptor dependent monocyte and macrophage responses results in additional anti-inflammatory activity for the present compounds beyond their effects on T cells. This activity is especially of value, for example, in the treatment of inflammatory diseases such as arthritis or inflammatory bowel disease. In particular, the present compounds are of value for the treatment of autoimmune glomerulonephritis and other instances of glomerulonephritis induced by deposition of immune complexes in the kidney that trigger Fc gamma receptor responses leading to kidney damage.

In addition, Src family kinases other than Lck, such as Lyn and Src, are important in the Fc epsilon receptor induced degranulation of mast cells and basophils that plays an important role in asthma, allergic rhinitis, and other allergic disease. Fc epsilon receptors are stimulated by IgE-antigen complexes. Compounds of the present invention inhibit the Fc epsilon induced degranulation responses, including in the basophil cell line RBL that does not express Lck. The ability to inhibit Fc epsilon receptor dependent mast cell and basophil responses results in additional anti-inflammatory activity for the present compounds beyond their effect on T cells. In particular, the present compounds are of value for the treatment of asthma, allergic rhinitis, and other instances of allergic disease.

The combined activity of the present compounds towards monocytes, macrophages, T cells, etc. may be of value in the treatment of any of the aforementioned disorders.

In a particular embodiment, the compounds of the present invention are useful for the treatment of the aforementioned exemplary disorders irrespective of their etiology, for example, for the treatment of transplant rejection, rheumatoid arthritis, multiple sclerosis, chronic obstructive pulmonary disease, inflammatory bowel disease, lupus, graft v. host disease, T-cell mediated hypersensitivity disease, psoriasis, Hashimoto's thyroiditis, Guillain-Barre syndrome, cancer, contact dermatitis, allergic disease such as allergic rhinitis, asthma, ischemic or reperfusion injury, or atopic dermatitis whether or not associated with PTK.

By virtue of their ability to inhibit HER1 and HER2 kinases, compounds of the present invention can also be used for the treatment of proliferative diseases, including psoriasis and cancer. The HER1 receptor kinase has been shown to be expressed and activated in many solid tumors including non-small cell lung, colorectal, and breast cancer.

Similarly, the HER2 receptor kinase has been shown to be overexpressed in breast, ovarian, lung and gastric cancer. Monoclonal antibodies that downregulate the abundance of the HER2 receptor or inhibit signaling by the HER1 receptor have shown anti-tumor effficacy in preclincal and clinical studies. It is therefore expected that inhibitors of the HER1 and HER2 kinases will have efficacy in the treatment of tumors that depend on signaling from either of the two receptors. These compounds are expected to have efficacy either as single agent or in combination with other chemotherapeutic agents such as placlitaxel (Taxol), doxorubicin hydrochloride (adriamycin), and cisplatin (Platinol). See the following documents and references cited therein: Cobleigh, M. A., Vogel, C. L., Tripathy, D., Robert, N. J., Scholl, S., Fehrenbacher, L., Wolter, J. M., Paton, V., Shak, S., Lieberman, G., and Slamon, D. J., “Multinational study of the efficacy and safety of humanized anti-HER2 monoclonal antibody in women who have HER2-overexpressing metastatic breast cancer that has progressed after chemotherapy for metastatic disease”, J. of Clin. Oncol. 17(9), p. 2639-2648 (1999); Baselga, J., Pfister, D., Cooper, M. R., Cohen, R., Burtness, B., Bos, M., D'Andrea, G., Seidman, A., Norton, L., Gunnett, K., Falcey, J., Anderson, V., Waksal, H., and Mendelsohn, J., “Phase I studies of anti-epidermal growth factor receptor chimeric antibody C225 alone and in combination with cisplatin”, J. Clin. Oncol. 18(4), p. 904-914 (2000).

The present invention also provides pharmaceutical compositions comprising at least one of the compounds of the present invention capable of treating a protein tyrosine kinase-associated disorder in an amount effective therefor, and a pharmaceutically acceptable vehicle or diluent. The compositions of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compounds of the present invention may be administered by any suitable means, for example, orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The present compounds may also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquify and/or dissolve in the rectal cavity to release the drug.

Exemplary compositions for topical administration include a topical carrier such as Plastibase (mineral oil gelled with polyethylene).

The effective amount of a compound of the present invention may be determined by one of ordinary skill in the art, and includes exemplary dosage amounts for an adult human of from about 0.1 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to protein tyrosine kinase-associated disorders.

When administered intravenously, the compounds of the present invention, including compounds of formula I, are preferably administered using the formulations of the invention. Generally, the compounds of the present invention, including compounds of formula (Ia), (Ib), and/or (Ic), are administered by IV infusion over a period of from about 10 minutes to about 3 hours, preferably about 30 minutes to about 2 hours, more preferably about 45 minutes to 90 minutes, and most preferably about 1 hour. Typically, the compounds are administered intravenously in a dose of from about 0.5 mg/m² to 65 mg/m², preferably about 1 mg/m² to 50 mg/m², more preferably about 2.5 mg/m² to 30 mg/m², and most preferably about 25 mg/m². One of ordinary skill in the art would readily know how to convert doses from mg/kg to mg/m2 given either or both the height and or weight of the patient (See, e.g., http://www.fda.gov/cder/cancer/animalframe.htm).

As discussed above, compounds of the present invention, including compounds of formula (Ia), (Ib), and/or (Ic), can be administered orally, intravenously, or both. In particular, the methods of the invention encompass dosing protocols such as once a day for 2 to 10 days, preferably every 3 to 9 days, more preferably every 4 to 8 days and most preferably every 5 days. In one embodiment there is a period of 3 days to 5 weeks, preferably 4 days to 4 weeks, more preferably 5 days to 3 weeks, and most preferably 1 week to 2 weeks, in between cycles where there is no treatment. In another embodiment the compounds of the present invention, including compounds of (Ia), (Ib), and/or (Ic), can be administered orally, intravenously, or both, once a day for 3 days, with a period of preferably 1 week to 3 weeks in between cycles where there is no treatment. In yet another embodiment the compounds of the present invention, including compounds of (Ia), (Ib), and/or (Ic), can be administered orally, intravenously, or both, once a day for 5 days, with a period of preferably 1 week to 3 weeks in between cycles where there is no treatment.

In one preferred embodiment the treatment cycle for administration of the compounds of the present invention, including compounds of (Ia), (Ib), and/or (Ic), is once daily for 5 consecutive days and the period between treatment cycles is from 2 to 10 days, preferably one week. In one embodiment, a compound of the present invention, for example, a compound of formula (Ia), (Ib), and/or (Ic), is administered once daily for 5 consecutive days, followed by 2 days when there is no treatment.

The compounds of the present invention, including compounds of formula (Ia), (Ib), and/or (Ic), can also be administered orally, intravenously, or both once every 1 to 10 weeks, preferably every 2 to 8 weeks, more preferably every 3 to 6 weeks, and even more preferably every 3 weeks.

In another method of the invention, the compounds of the present invention, including compounds of formula (Ia), (Ib), and/or (Ic), are administered in a 28 day cycle wherein the compounds are intravenously administered on days 1, 7, and 14 and orally administered on day 21. Alternatively, the compounds of the present invention, including compounds of formula (Ia), (Ib), and/or (Ic), are administered in a 28 day cycle wherein the compound of formula (Ia), (Ib), and/or (Ic) are orally administered on day 1 and intravenously administered on days 7, 14, and 28.

According to the methods of the invention, the compounds of the present invention, including compounds of formula (Ia), (Ib), and/or (Ic), are administered until the patient shows a response, for example, a reduction in tumor size, or until dose limiting toxicity is reached.

The compounds of the present invention may be employed alone or in combination with each other and/or other suitable therapeutic agents useful in the treatment of protein tyrosine kinase-associated disorders such as PTK inhibitors other than those of the present invention, antiinflammatories, antiproliferatives, chemotherapeutic agents, immunosuppressants, anticancer agents and cytotoxic agents.

Exemplary such other therapeutic agents include the following: cyclosporins (e.g., cyclosporin A), CTLA4-Ig, antibodies such as anti-ICAM-3, anti-IL-2 receptor (Anti-Tac), anti-CD45RB, anti-CD2, anti-CD3 (OKT-3), anti-CD4, anti-CD80, anti-CD86, monoclonal antibody OKT3, agents blocking the interaction between CD40 and gp39, such as antibodies specific for CD40 and/or gp39 (i.e., CD154), fusion proteins constructed from CD40 and gp39 (CD40Ig and CD8gp39), inhibitors, such as nuclear translocation inhibitors, of NF-kappa B function, such as deoxyspergualin (DSG), non-steroidal antiinflammatory drugs (NSAIDs) such as ibuprofen, steroids such as prednisone or dexamethasone, gold compounds, antiproliferative agents such as methotrexate, FK506 (tacrolimus, Prograf), mycophenolate mofetil, cytotoxic drugs such as azathiprine and cyclophosphamide, TNF-□ inhibitors such as tenidap, anti-TNF antibodies or soluble TNF receptor such as etanercept (Enbrel), rapamycin (sirolimus or Rapamune), leflunimide (Arava), and cyclooxygenase-2 (COX-2) inhibitors such as celecoxib (Celebrex) and rofecoxib (Vioxx), or derivatives thereof, and the PTK inhibitors disclosed in the following U.S. Patent Applications, incorporated herein by reference in their entirety: Ser. No. 60/056,770, filed Aug. 25, 1997 (Attorney Docket No. QA202*), Ser. No. 60/069,159, filed Dec. 9, 1997 (Attorney Docket No. QA202a*), Ser. No. 09/097,338, filed Jun. 15, 1998 (Attorney Docket No. QA202b), Ser. No. 60/056,797, filed Aug. 25, 1997 (Attorney Docket No. QA205*), Ser. No. 09/094,797, filed Jun. 15, 1998 (Attorney Docket No. QA205a), Ser. No. 60/065,042, filed Nov. 10, 1997 (Attorney Docket No. QA207*), Ser. No. 09/173,413, filed Oct. 15, 1998, (Attorney Docket No. QA207a), Ser. No. 60,076,789, filed Mar. 4, 1998 (Attorney Docket No. QA208*), and Ser. No. 09,262,525, filed Mar. 4, 1999 (Attorney Docket No. QA208a). See the following documents and references cited therein: Hollenbaugh, D., Douthwright, J., McDonald, V., and Aruffo, A., “Cleavable CD40Ig fusion proteins and the binding to sgp39”, J. Immunol. Methods (Netherlands), 188(1), p. 1-7 (Dec. 15, 1995); Hollenbaugh, D., Grosmaire, L. S., Kullas, C. D., Chalupny, N. J., Braesch-Andersen, S., Noelle, R. J., Stamenkovic, I., Ledbetter, J. A., and Aruffo, A., “The human T cell antigen gp39, a member of the TNF gene family, is a ligand for the CD40 receptor: expression of a soluble form of gp39 with B cell co-stimulatory activity”, EMBO J (England), 11(12), p 4313-4321 (December 1992); and Moreland, L. W. et al., “Treatment of rheumatoid arthritis with a recombinant human tumor necrosis factor receptor (p75)-Fc fusion protein, New England J. of Medicine, 337(3), p. 141-147 (1997).

Exemplary classes of anti-cancer agents and cytotoxic agents include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines (e.g., daunorubicin, doxorubicin), cytarabine (ara-C; Cytosar-U®); 6-thioguanine (Tabloid®), mitoxantrone (Novantrone®) and etoposide (VePesid®), amsacrine (AMSA), and all-trans retinoic acid (ATRA), bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compounds of the invention may also be used in conjunction with radiation therapy.

Representative examples of these classes of anti-cancer and cytotoxic agents include, but are not limited to, mechlorethamine hydrochlordie, cyclophosphamide, chlorambucil, melphalan, ifosfamide, busulfan, carmustin, lomustine, semustine, streptozocin, thiotepa, dacarbazine, methotrexate, thioguanine, mercaptopurine, fludarabine, pentastatin, cladribin, cytarabine, fluorouracil, doxorubicin hydrochloride, daunorubicin, idarubicin, bleomycin sulfate, mitomycin C, actinomycin D, safracins, saframycins, quinocarcins, discodermolides, vincristine, vinblastine, vinorelbine tartrate, etoposide, teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, gemcitabine hydrochloride, altretamine, and topoteca and any analogs or derivatives thereof.

Preferred members of these classes include, but are not limited to paclitaxel, cisplatin, carboplatin, doxorubicin, carminomycin, daunorubicin, aminopterin, methotrexate, methopterin, mitomycin C, ecteinascidin 743, porfiromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine, and leurosine.

In the field of medical oncology it is normal practice to use a combination of different forms of treatment to treat each patient with cancer. In medical oncology the other component(s) of such conjoint treatment in addition to the antiproliferative treatment defined herein before may be: surgery, radiotherapy or chemotherapy. Such chemotherapy may cover three main categories of therapeutic agent:
(i) antiangiogenic agents that work by different mechanisms from those defined hereinbefore (for example, linomide, inhibitors of integrin αvβ3 function, angiostatin, razoxane);
(ii) cytostatic agents such as antiestrogens (for example, tamoxifen, toremifene, raloxifene, droloxifene, iodoxifene), progestogens (for example megestrol acetate), aromatase inhibitors (for example, anastrozole, letrozole, borazole, exemestane), antihormones, antiprogestogens, antiandrogens (for example, flutamide, nilutamide, bicalutamide, cyproterone acetate), LHRH agonists and antagonists (for example, gosereline acetate, leuprolide), inhibitors of testosterone 5α-dihydroreductase (for example, finasteride), farnesyltransferase inhibitors, anti-invasion agents (for example, metalloproteinase inhibitors like marimastat and inhibitors of urokinase plasminogen activator receptor function) and inhibitors of growth factor function, (such growth factors include for example, EGF, FGF, platelet derived growth factor and hepatocyte growth factor such inhibitors include growth factor antibodies, growth factor receptor antibodies such as Avastin® (bevacizumab) and Erbitux® (cetuximab); tyrosine kinase inhibitors and serine/threonine kinase inhibitors such as Gleevec® (imatinib mesylate)); and
(iii) antiproliferative/antineoplastic drugs and combinations thereof, as used in medical oncology, such as antimetabolites (for example, antifolates like methotrexate, fluoropyrimidines like 5-fluorouracil, purine and adenosine analogues, cytosine arabinoside); Intercalating antitumour antibiotics (for example, anthracyclines like doxorubicin, daunomycin, epirubicin and idarubicin, mitomycin-C, dactinomycin, mithramycin); platinum derivatives (for example, cisplatin, carboplatin); alkylating agents (for example, nitrogen mustard, melphalan, chlorambucil, busulphan, cyclophosphamide, ifosfamide nitrosoureas, thiotepa; antimitotic agents (for example, vinca alkaloids like vincristine, vinblastine and vinflunine, and taxoids like Taxol® (paclitaxel), Taxotere® (docetaxel) and newer microbtubule agents such as epothilone analogs, discodermolide analogs, and eleutherobin analogs); topoisomerase inhibitors (for example, epipodophyllotoxins like etoposide and teniposide, amsacrine, topotecan); cell cycle inhibitors (for example, flavopyridols); biological response modifiers and proteasome inhibitors such as Velcade® (bortezomib).

The compounds of the present invention may be used in combination with anti-cancer compounds such as fentanyl, doxorubicin, interferon alfa-n3, palonosetron dolasetron anastrozole, exemestane, bevacizumab, bicalutamide, cisplatin, dacarbazine, cytarabine, clonidine, epirubicin, levamisole, toremifene, fulvestrant, letrozole, tamsulosin, gallium nitrate, trastuzumab, altretamine, hydroxycarbamide, ifosfamide, interferon alfacon-1, gefitinib, granisetron, leuprorelin, dronabinol, megestrol, pethidine, promethazine, morphine, vinorelbine, pegfilgrastim, filgrastim, nilutamide, thiethylperazine, leuprorelin, pegaspargase, muromonab-CD3, porfimer sodium, cisplatin, abarelix, capromab, samarium SM153 lexidronam, paclitaxel, docetaxel, etoposide, triptorelin, valrubicin, nofetumomab merpentan technetium 99 m Tc, vincristine, capecitabine, strptozocin, and ondansetron.

In another embodiment of the invention a compounds of the present invention are administered in conjunction with at least one anti-neoplastic agent or anti cancer agent. As used herein, the phrase “anti-neoplastic agent” or “anti-cancer agent” is synonymous with “chemotherapeutic agent” and/or “anti-proliferative agent” and refers to compounds that prevent cancer, or hyperproliferative cells from multiplying. Anti-proliferative agents prevent cancer cells from multiplying by: (1) interfering with the cell's ability to replicate DNA and (2) inducing cell death and/or apoptosis in the cancer cells.

The term “anticancer” agent includes any known agent that is useful for the treatment of cancer including the following: 17α-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, estramustine, medroxyprogesteroneacetate, leuprolide, flutamide, toremifene, Zoladex; matrix metalloproteinase inhibitors; VEGF inhibitors, such as anti-VEGF antibodies (Avastin) and small molecules such as ZD6474, AZD-2171, SU6668; Vatalanib, BAY-43-9006, SU11248, CP-547632, and CEP-7055; Her 1 and Her 2 inhibitors including anti-Her2 antibodies (Herceptin); EGFR inhibitors including gefitinib, erlotinib, ABX-EGF, EMD72000, 11F8, and cetuximab; Eg5 inhibitors, such as SB-715992, SB-743921, and MKI-833; pan Her inhibitors, such as canertinib, EKB-569, CI-1033, AEE-788, XL-647, mAb 2C4, and GW-572016; Src kinase inhibitors such as BMS-354825, AZD-0530, SKI-606, and AP-23464; Bcr-Abl inhibitors such as imatinib and AMN107; Casodex® (bicalutamide, Astra Zeneca), Tamoxifen; MEK-1 kinase inhibitors, MAPK kinase inhibitors, PI3 kinase inhibitors; Met inhibitors, Aurora kinase inhibitors, PDGF inhibitors; anti-angiogenic and antivascular agents which, by interrupting blood flow to solid tumors, render cancer cells quiescent by depriving them of nutrition; castration, which renders androgen dependent carcinomas non-proliferative; IGF1R inhibitors such as those disclosed in US2004/44203A1, inhibitors of non-receptor and receptor tyrosine kinases; inhibitors of integrin signaling; tubulin acting agents such as vinblastine, vincristine, vinorelbine, vinflunine, paclitaxel, docetaxel, 7-O-methylthiomethylpaclitaxel, 4-desacetyl-4-methylcarbonatepaclitaxel, 3′-tert-butyl-3′-N-tert-butyloxycarbonyl-4-deacetyl-3′-dephenyl-3′-N-debenzoyl-4-O-methoxycarbonyl-paclitaxel, C-4 methyl carbonate paclitaxel, epothilone A, epothilone B, epothilone C, epothilone D, desoxyepothilone A, desoxyepothilone B, ixabepilone, [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione, and derivatives thereof; CDK inhibitors, antiproliferative cell cycle inhibitors, epidophyllotoxin, etoposide, VM-26; antineoplastic enzymes, e.g., topoisomerase I inhibitors, camptothecin, topotecan, SN-38; procarbazine; mitoxantrone; platinum coordination complexes such as cisplatin, carboplatin and oxaliplatin; biological response modifiers; growth inhibitors; antihormonal therapeutic agents; leucovorin; tegafur; antimetabolites such as purine antagonists (e.g. 6-thioguanine and 6-mercaptopurine; glutamine antagonists, e.g. DON (AT-125; d-oxo-norleucine); ribonucleotide reductase inhibitors; mTOR inhibitors; and haematopoietic growth factors.

Additional cytotoxic agents include, cyclophosphamide, doxorubicin, daunorubicin, mitoxanthrone, melphalan, hexamethyl melamine, thiotepa, cytarabin, idatrexate, trimetrexate, dacarbazine, L-asparaginase, bicalutamide, leuprolide, pyridobenzoindole derivatives, interferons, and interleukins.

In cases where it is desirable to render aberrantly proliferative cells quiescent in conjunction with or prior to treatment with the chemotherapeutic methods of the invention, hormones and steroids (including synthetic analogs): 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, hlorotrianisene, Hydroxyprogesterone, Aminoglutethimide, Estramustine, Medroxyprogesteroneacetate, Leuprolide, Flutamide, Toremifene, Zoladex can also be administered to the patient.

Also suitable for use in the combination chemotherapeutic methods of the invention are antiangiogenics such as matrix metalloproteinase inhibitors, and other VEGF inhibitors, such as anti-VEGF antibodies and small molecules such as ZD6474 and SU6668 are also included. Anti-Her2 antibodies from Genetech may also be utilized. A suitable EGFR inhibitor is EKB-569 (an irreversible inhibitor). Also included are Imclone antibody C225 immunospecific for the EGFR, and src inhibitors. Also suitable for use as an antiproliferative cytostatic agent is Casodex™ which renders androgen-dependent carcinomas non-proliferative. Yet another example of a cytostatic agent is the antiestrogen Tamoxifen which inhibits the proliferation or growth of estrogen dependent breast cancer. Inhibitors of the transduction of cellular proliferative signals are cytostatic agents. Examples are epidermal growth factor inhibitors, Her-2 inhibitors, MEK-1 kinase inhibitors, MAPK kinase inhibitors, PI3 inhibitors, Src kinase inhibitors, and PDGF inhibitors.

As mentioned, certain anti-proliferative agents are anti-angiogenic and antivascular agents and, by interrupting blood flow to solid tumors, render cancer cells quiescent by depriving them of nutrition. Castration, which also renders androgen dependent carcinomas non-proliferative, may also be utilized. Starvation by means other than surgical disruption of blood flow is another example of a cytostatic agent. A particular class of antivascular cytostatic agents is the combretastatins. Other exemplary cytostatic agents include MET kinase inhibitors, MAP kinase inhibitors, inhibitors of non-receptor and receptor tyrosine kinases, inhibitors of integrin signaling, and inhibitors of insulin-like growth factor receptors.

The compounds of the present invention may be useful in combination with BCR-ABL inhibitors such as, but not limited to, Gleevec® (imatinib, STI-571) or AM-107.

The compounds of the present invention may be useful in combination with anti-cancer compounds such as fentanyl, doxorubicin, interferon alfa-n3, palonosetron dolasetron anastrozole, exemestane, bevacizumab, bicalutamide, cisplatin, dacarbazine, cytarabine, clonidine, epirubicin, levamisole, toremifene, fulvestrant, letrozole, tamsulosin, gallium nitrate, trastuzumab, altretamine, hydroxycarbamide, ifosfamide, interferon alfacon-1, gefitinib, granisetron, leuprorelin, dronabinol, megestrol, pethidine, promethazine, morphine, vinorelbine, pegfilgrastim, filgrastim, nilutamide, thiethylperazine, leuprorelin, pegaspargase, muromonab-CD3, porfimer sodium, cisplatin, abarelix, capromab, samarium SM153 lexidronam, paclitaxel, docetaxel, etoposide, triptorelin, valrubicin, nofetumomab merpentan technetium 99 m Tc, vincristine, capecitabine, strptozocin, and ondansetron.

The above other therapeutic agents, when employed in combination with the compounds of the present invention, may be used, for example, in those amounts indicated in the Physicians' Desk Reference (PDR) or as otherwise determined by one of ordinary skill in the art.

It is desirable to find new compounds with improved pharmacological characteristics compared with known protein tyrosine kinase inhibitors. It is desirable to find compounds with advantageous and improved characteristics in one or more of the following categories: (a) pharmaceutical properties (i.e. solubility, permeability, amenability to sustained release formulations); (b) dosage requirements (e.g., lower dosages and/or once-daily dosing); (c) factors which decrease blood concentration peak-to-trough characteristics (i.e. clearance and/or volume of distribution); (d) factors that increase the concentration of active drug at the receptor (i.e. protein binding, volume of distribution); (e) factors that decrease the liability for clinical drug-drug interactions; (f) factors that decrease the potential for adverse side-effects (i.e. pharmacological selectivity beyond G protein-coupled receptors, potential chemical or metabolic reactivity, limited CNS penetration) (g) factors that improve manufacturing costs or feasibility (i.e. difficulty of synthesis, number of chiral centers, chemical stability, ease of handling).

The compounds of the present invention are active as protein kinase inhibitors either in their current composition or as prodrugs for the compound of Formula A.

The following assays can be employed in ascertaining the degree of activity of a compound (“test compound”) as a PTK inhibitor. Compounds described in the following Examples have been tested in one or more of these assays and/or act as prodrugs for the compound of formula A which has been tested in one or more of these assays, and have shown activity.

Enzyme Assay Using Lck, Fyn, Lyn, Hck, Fgr, Src, Blk or Yes

The following assay has been carried out using the protein tyrosine kinases Lck, Fyn, Lyn, Hck, Fgr, Src, Blk and Yes.

The protein tyrosine kinase of interest is incubated in kinase buffer (20 mM MOPS, pH 7, 10 mM MgCl₂) in the presence of the test compound. The reaction is initiated by the addition of substrates to the final concentration of 1 μM ATP, 3.3 μCi/ml [33P] gamma-ATP, and 0.1 mg/ml acid denatured enolase (prepared as described in Cooper, J. A., Esch, F. S., Taylor, S. S., and Hunter, T., “Phosphorylation sites in enolase and lactate dehydrogenase utilized by tyrosine protein kinases in vivo and in vitro”, J. Biol. Chem., 259, 7835-7841 (1984)). The reaction is stopped after 10 minutes by the addition of 10% trichloroacetic acid, 100 mM sodium pyrophosphate followed by 2 mg/ml bovine serum albumin. The labeled enolase protein substrate is precipitated at 4 degrees, harvested onto Packard Unifilter plates and counted in a Topcount scintillation counter to ascertain the protein tyrosine kinase inhibitory activity of the test compound (activity inversely proportional to the amount of labeled enolase protein obtained). The exact concentration of reagents and the amount of label can be varied as needed.

This assay is advantageous as it employs an exogenous substrate (enolase) for more accurate enzyme kinetics, and can be conducted in a 96-well format that is readily automated. In addition, His-tagged protein tyrosine kinases (described below) offer much higher production yields and purity relative to GST-protein tyrosine kinase fusion protein.

The protein tyrosine kinase may be obtained from commercial sources or by recombinant methods described herewith. For the preparation of recombinant Lck, human Lck was prepared as a His-tagged fusion protein using the Life Technologies (Gibco) baculovirus vector pFastBac Hta (commercially available) in insect cells. A cDNA encoding human Lck isolated by PCR (polymerase chain reaction) was inserted into the vector and the protein was expressed using the methods described by the manufacturer. The Lck was purified by affinity chromatography. For the production of Lck in insect cells using baculovirus, see Spana, C., O'Rourke, E. C., Bolen, J. B., and Fargnoli, J., “Analysis of the tyrosine kinase p56ck expressed as a glutathione S-transferase protein in Spodoptera frugiperda cells,” Protein expression and purification, Vol. 4, p. 390-397 (1993). Similar methods may be used for the recombinant production of other Src-family kinases.

Enzyme Assay Using HER1 or HER2

Compounds of interest were assayed in a kinase buffer that contained 20 mM Tris.HCl, pH 7.5, 10 mM MnCl₂, 0.5 mM dithiothreitol, bovine serum albumin at 0.1 mg/ml, poly(glu/tyr, 4:1) at 0.1 mg/ml, 1 μM ATP, and 4 μCi/ml [gamma-³³P]ATP. Poly(glu/tyr, 4:1) is a synthetic polymer that serves as a phosphoryl acceptor and is purchased from Sigma Chemicals. The kinase reaction is initiated by the addition of enzyme and the reaction mixtures were incubated at 26° C. for 1 h. The reaction is terminated by the addition of EDTA to 50 mM and proteins are precipitated by the addition of trichloroacetic acid to 5%. The precipitated proteins are recovered by filtration onto Packard Unifilter plates and the amount of radioactivity incorporated is measured in a Topcount scintillation counter.

For the preparation of recombinant HER1, the cytoplasmic sequence of the receptor were expressed in insect cells as a GST fusion protein, which was purified by affinity chromatography as described above for Lck. The cytoplasmic sequence of HER2 was subcloned into the baculovirus expression vector pBlueBac4 (Invitrogen) and was expressed as an untagged protein in insect cells. The recombinant protein was partially purified by ion-exchange chromatography.

Cell Assays

Cellular Tyrosine Phosphorylation

Jurkat T cells are incubated with the test compound and then stimulated by the addition of antibody to CD3 (monoclonal antibody G19-4). Cells are lysed after 4 minutes or at another desired time by the addition of a lysis buffer containing NP-40 detergent. Phosphorylation of proteins is detected by anti-phosphotyrosine immunoblotting. Detection of phosphorylation of specific proteins of interest such as ZAP-70 is detected by immunoprecipitation with anti-ZAP-70 antibody followed by anti-phosphotyrosine immunoblotting. Such procedures are described in Schieven, G. L., Mittler, R. S., Nadler, S. G., Kirihara, J. M., Bolen, J. B., Kanner, S. B., and Ledbetter, J. A., “ZAP-70 tyrosine kinase, CD45 and T cell receptor involvement in UV and H₂O₂ induced T cell signal transduction”, J. Biol. Chem., 269, 20718-20726 (1994), and the references incorporated therein. The Lck inhibitors inhibit the tyrosine phosphorylation of cellular proteins induced by anti-CD3 antibodies.

For the preparation of G19-4, see Hansen, J. A., Martin, P. J., Beatty, P. G., Clark, E. A., and Ledbetter, J. A., “Human T lymphocyte cell surface molecules defined by the workshop monoclonal antibodies,” in Leukocyte Typing I, A. Bernard, J. Boumsell, J. Dausett, C. Milstein, and S. Schlossman, eds. (New York: Springer Verlag), p. 195-212 (1984); and Ledbetter, J. A., June, C. H., Rabinovitch, P. S., Grossman, A., Tsu, T. T., and Imboden, J. B., “Signal transduction through CD4 receptors: stimulatory vs. inhibitory activity is regulated by CD4 proximity to the CD3/T cell receptor”, Eur. J. Immunol., 18, 525 (1988).

Calcium Assay

Lck inhibitors block calcium mobilization in T cells stimulated with anti-CD3 antibodies. Cells are loaded with the calcium indicator dye indo-1, treated with anti-CD3 antibody such as the monoclonal antibody G19-4, and calcium mobilization is measured using flow cytometry by recording changes in the blue/violet indo-1 ratio as described in Schieven, G. L., Mittler, R. S., Nadler, S. G., Kirihara, J. M., Bolen, J. B., Kanner, S. B., and Ledbetter, J. A., “ZAP-70 tyrosine kinase, CD45 and T cell receptor involvement in UV and H₂O₂ induced T cell signal transduction”, J. Biol. Chem., 269, 20718-20726 (1994), and the references incorporated therein.

Proliferation Assays

Lck inhibitors inhibit the proliferation of normal human peripheral blood T cells stimulated to grow with anti-CD3 plus anti-CD28 antibodies. A 96 well plate is coated with a monoclonal antibody to CD3 (such as G19-4), the antibody is allowed to bind, and then the plate is washed. The antibody bound to the plate serves to stimulate the cells. Normal human peripheral blood T cells are added to the wells along with test compound plus anti-CD28 antibody to provide co-stimulation. After a desired period of time (e.g., 3 days), the [3H]-thymidine is added to the cells, and after further incubation to allow incorporation of the label into newly synthesized DNA, the cells are harvested and counted in a scintillation counter to measure cell proliferation.

SRC Kinase

The biochemical kinase assay to quantitate the inhibition of kinase activity by kinase inhibitors were performed in vitro in 96-well microtiter plates. All kinase inhibitors were dissolved in 100% DMSO and diluted into 2× the final concentration with PBS/1% DMSO prior to assay. Kinase reaction consisted of 5 ng of baculovirus expressed GST-SRC, 1.5 mM poly(Glu/Tyr) (Sigma), 0.3 mM ATP, and 0.15 mCi [g-33P]ATP in 50 ml kinase buffer (50 mM Tris, pH 7.4, 2 mM dithiothreitol (DTT), 0.1 mg/ml BSA, 0.3 mM MnCl2). The reaction mixture was incubated at 28° C. for 1 hour. The reaction was terminated by adding 10 ml of stopping buffer consisting of 2.5 mg/ml BSA and 300 mM EDTA followed by immediate precipitation with 110 ml of 10% TCA on ice for 30 min. The precipitates were transferred to a 96-well UniFilter GF/C plate. The amount of the phosphorylated synthetic substrate was quantitated using a TopCount 96-well liquid scintillation counter (PerkinElmer Life Sciences Inc, Boston, Mass.). Dose-response curves were generated to determine the concentration of the inhibitors required to inhibit 50% of kinase activity (IC50). IC50 values were derived by non-linear regression analysis and have a coefficient of variance=16% (SD/mean, n=3). The reported IC50 value was the average from at least three separate experiments.

LCK Kinase

The same procedure as that of SRC kinase detailed above was applied to the LCK kinase assay, except that the reaction consisted of 20 ng baculovirus expressed GST-LCK protein.

YES Kinase

The same procedure as that of SRC kinase detailed above was applied to the YES kinase assay, except that the reaction consisted of 10 ng baculovirus expressed GST-YES protein.

FYN Kinase

The same procedure as that of SRC kinase detailed above was applied to the LCK kinase assay, except that the reaction consisted of 20 ng baculovirus expressed GST-FYN protein.

BCR-ABL Kinase

The same procedure as that of SRC kinase detailed above was applied to the BCR-ABL kinase assay, except that the reaction consisted of 250 ng baculovirus expressed GST-BCR-ABL protein.

c-KIT Kinase

The biochemical assay to determine inhibition of c-KIT kinase activity was performed as described in section 3.1.2.1, with the exception that each reaction mixture contained 250 ng of recombinant GST-c-KIT protein purified from Sf9 insect cells. The GST-c-KIT protein contains the entire cytoplasmic sequence of c-KIT. The mixture contained also 1.5 mM poly (Glu/Tyr) (Sigma), 1 M ATP, and 0.15 mCi[g-33P]ATP in 50 ml kinase buffer (50 mM Tris, pH 7.7, 2 mM DTT, 0.1 mg/ml BSA, 5 mM MgCl2). Incorporation of radioactive phosphate and the determination of IC50 values were also carried out as described above.

PDGF Receptor Kinase

The PDGFR-b human receptor tyrosine kinase was assayed using the synthetic polymer poly(Glu4/Tyr) (Sigma Chemicals) as a phosphoacceptor substrate. Each reaction mixture consisted of a total volume of 50 ml and contained 200 ng of baculovirus expressed enzyme, 64 mg/ml poly(Glu4/Tyr), 3.6 mM of ATP, and 0.7 mCi of [g-33P]ATP. The mixture also contained 20 mM HEPES, pH 7.0, 5 mM MnCl2, 150 mM NaCl, 0.5 mM DDT, and 25 mg/ml bovine serum albumin (BSA). The reaction mixtures were incubated at 27° C. for 60 minutes and kinase activity was determined by quantitation of the amount of radioactive phosphate transferred to the poly(Glu4/Tyr) substrate. Incorporation was measured by the addition of cold trichloroacetic acid. Precipitates were collected onto GF/C UniFilter plates (Packard Instrument Co., Meriden, Conn.) using a Filtermate universal harvester and quantitated using a TopCount 96-well liquid scintillation counter (Packard Instrument Co., Meriden, Conn.). Compounds were dissolved in dimethylsulfoxide (DMSO) to a concentration of 10 mM and were evaluated at six concentrations diluted four-fold, each in triplicate. The final concentration of DMSO in the kinase assays was 0.5%, which was shown to have no effect on kinase activity. IC50 values were derived by non-linear regression analysis and have a coefficient of variance (SD/mean, n=6)=10%.

EPHA2 Receptor Kinase

The biochemical assay to determine inhibition of EPHA2 kinase activity was performed as described above, with the exception that each reaction mixture contained 100 ng of recombinant GST-EPHA2 protein purified from Sf9 insect cells. The GST-EphA2 protein consists of the entire cytoplasmic sequence of EPHA2 fused to the c-terminus of GST. The mixture contained also 1.5 mM poly (Glu/Tyr) (Sigma), 1 mM ATP, and 0.15 mCi[g-33P]ATP in 50 ml kinase buffer (50 mM Tris, pH 7.7, 2 mM DTT, 0.1 mg/ml BSA, 5 mM MgCl2). Incorporation of radioactive phosphate and the determination of IC50 values were also carried out as described above.

### Methods of Preparation

The compounds of the formula (Ia), (Ib), and/or (Ic) may be prepared by methods such as those illustrated in the following Schemes 1-4. Solvents, temperatures, pressures, and other reaction conditions may readily be selected by one of ordinary skill in the art. All documents cited are incorporated herein by reference in their entirety. Starting materials are commercially available or readily prepared by one of ordinary skill in the art. Constituents of compounds are as defined elsewhere in the specification or as specifically defined in a scheme.

The present invention is directed to prodrugs of compound (A). Various forms of prodrugs are well known in the art. For examples of such prodrug delivery derivatives, see:

a) Design of Prodrugs, H. Bundgaard (editor), Elsevier (1985);

b) Methods in Enzymology, K. Widder et al. (editors), Academic Press, Vol. 112, 309-396 (1985);

c) A Textbook of Drug Design and Development, Krosgaard-Larsen and H. Bundgaard (editors), Chapter 5, “Design and Application of Prodrugs,” 113-191 (1991);

d) H. Bundgaard, Advanced Drug Delivery Reviews, 8, 1-38 (1992);

e) H. Bundgaard, J. of Pharm. Sciences, 77, 285 (1988); and

f) N. Kakeya et al., Chem. Pharm. Bull., 32 692 (1984).

Various ester and carbonate prodrugs can be prepared by reacting alcohol 1 with acyl halides in the presence of base such as triethylamine and a catalytic amount of 4-dimethylaminopyridine. Alternatively, esters can be obtained using the various acids in the presence of activating agents such as EDCI and 4-dimethylaminopyridine in a solvent like DMF or THF (Scheme 1).

Phosphate bis-sodium salt 9 can be prepared by coupling of alcohol 1 with di-t-butylphosphoramidite followed by mCPBA oxidation, deprotection of the t-butyls with TFA and salt formation using aq. NaOH solution (Scheme 2).

Carbamate prodrugs such as 11 or 12 can be prepared by reacting the silyl protected compound 10 with alkyl chloroformate in the presence of base such as pyridine and a catalytic amount of 4-dimethylaminopyridine. Deprotection of the silyl group of the resulting carbamate with tetra-n-butylammonium fluoride can provide the carbamate prodrugs (Scheme 3).

Ester prodrugs via the methylene linker on the nitrogen atom of the aminothiazole moiety can be prepared using the appropriate chloromethyloxycarbonyl alkyl or aryl derivatives in the presence of base such as cesium carbonate or sodium hydride followed by deprotection of the silyl group. Depending on the substrate alkylation can produce either the coupling adduct on the amino group such as analogues 13 and 14 as a very major or an adduct on the thiazole nitrogen such as 15 as a very major. In some cases coupling reaction provided a mixture of two region-isomers like 16 and 17 in a 5:3 ratio (Scheme 3).

Dioxalenone prodrug like 19 can be prepared by coupling of silyl ether 10 with 4-bromomethy-5-methyldioxalenone followed by deprotection of the silyl group to obtain prodrug 19 (Scheme 4).

The following Examples illustrate embodiments, and are not intended to limit the scope of the claims. Abbreviations employed in the Examples are defined below. Compounds of the Examples are identified by the example and step in which they are prepared (for example, “1A” denotes the title compound of step A of Example 1), or by the example only where the compound is the title compound of the example (for example, “2” denotes the title compound of Example 2).

### ABBREVIATIONS

aq.=aqueous
conc.=concentrated
DMSO=dimethylsulfoxide
EtOAc=ethyl acetate
Et₂O=diethyl ether
h=hours
HATU═N-[dimethylamino-1H-1,2,3-triazolo-[4,5-b]pyridin-1-yl methylene]-N-methyl methanaminium hexafluorophosphate N-oxide
MeOH=methanol
MOPS=4-morpholine-propanesulfonic acid
MS=mass spectrometry
Ret Time=retention time
RT=room temperature
satd.=saturated
TFA=trifluoroacetic acid
THF=tetrahydrofuran
DMF═N,N-dimethylformamide

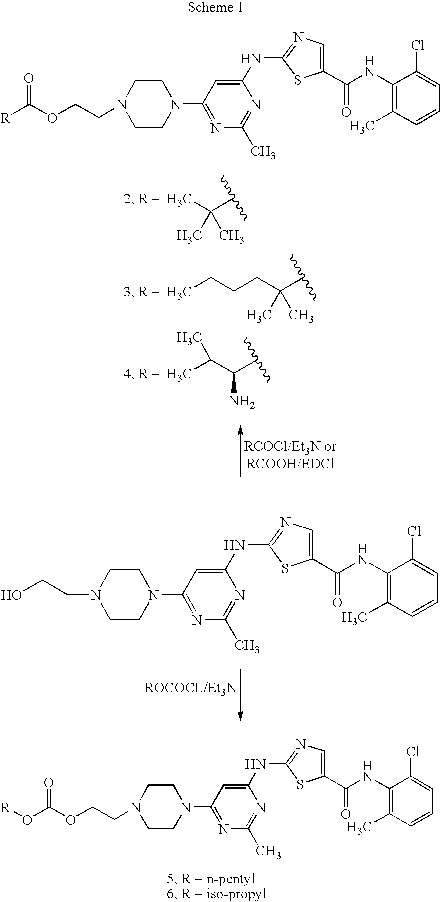

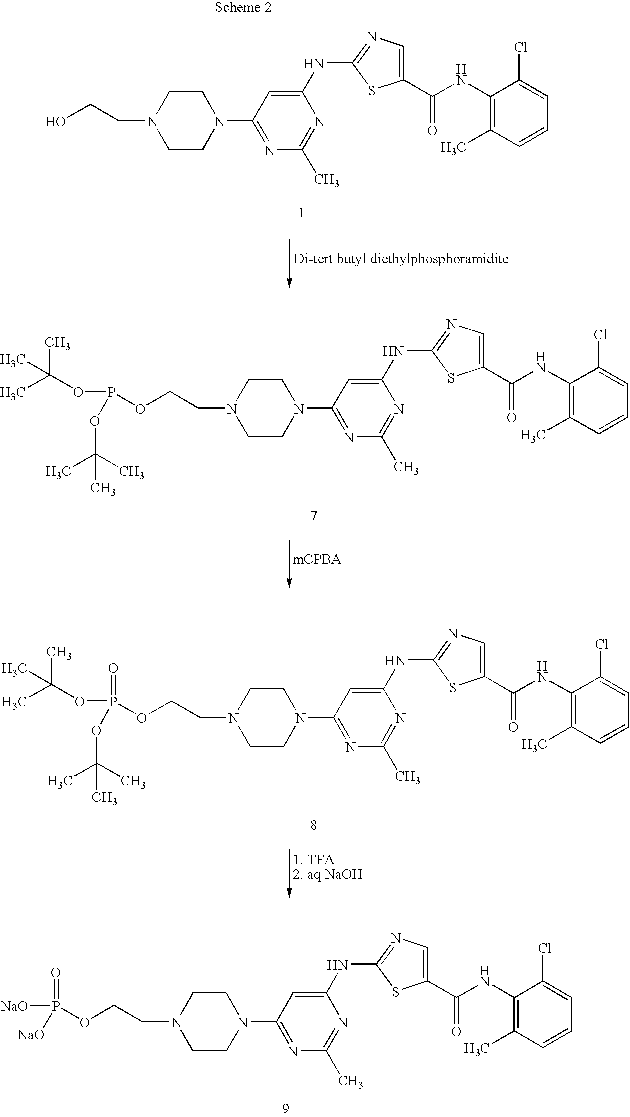

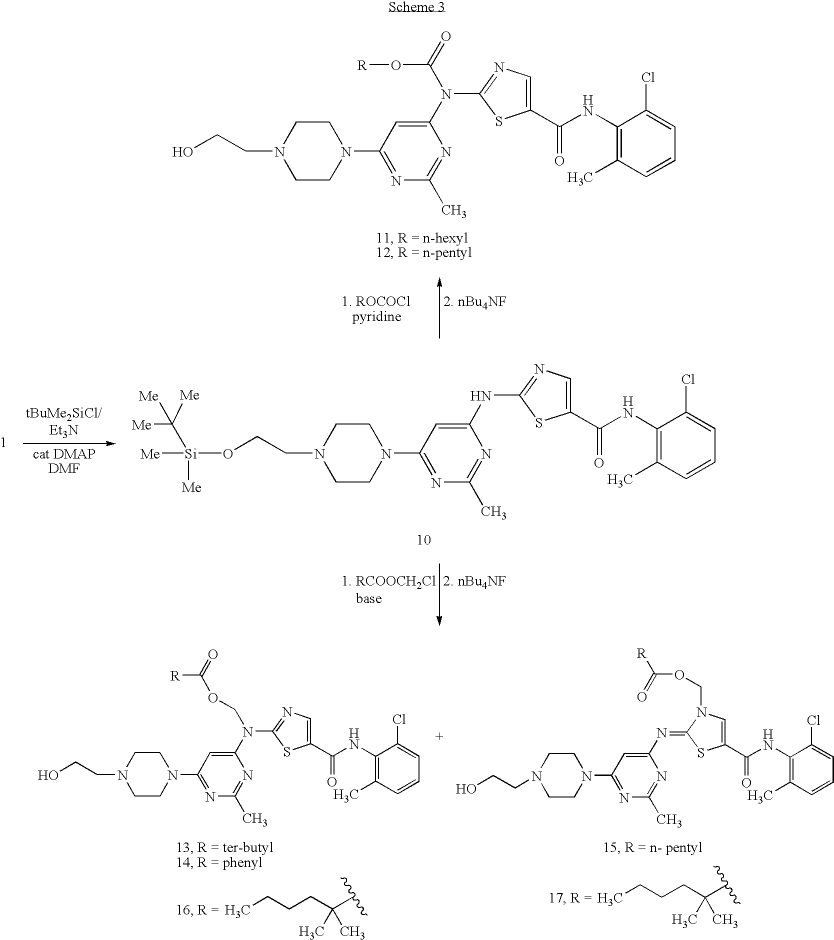

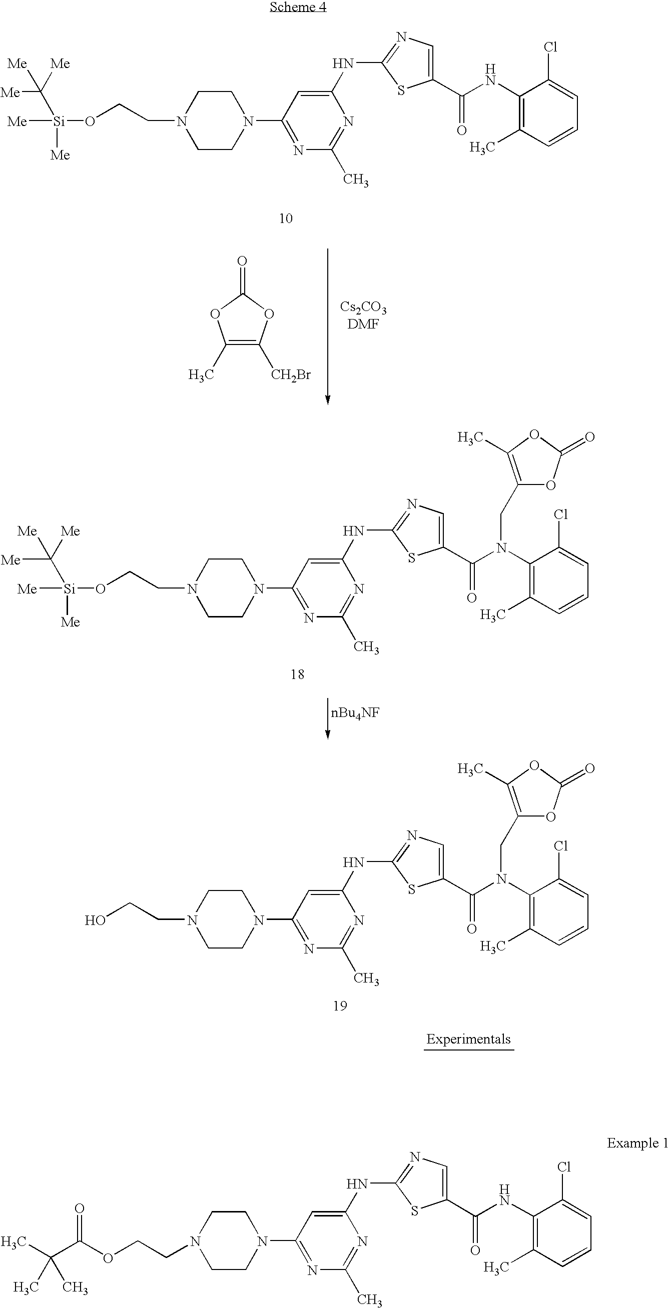

### 2-(4-(6-(5-((2-Chloro-6-methylphenyl)carbamoyl)thiazol-2-ylamino)-2-methyl pyrimidin-4-yl)piperazin-1-yl)ethyl pivalate (2)

To a stirred mixture of 1 (10.0 g, 20.4 mmole), Et₃N (5.8 mL, 41.6 mmole) and dimethylaminopyridine (0.40 g, 3.27 mmole)) in DMF (60 mL) at room temperature under nitrogen was added trimethylacetyl chloride (4.2 mL, 34.1 mmole). The resultant mixture was stirred at room temperature for 2 days. It was added with water, and the solid precipitate was collected which was washed with water and Et₂O. The crude solid was re-crystallized twice from DMF-H₂O, and dried in vacuo at 30° C. over MgSO₄ overnight to pivalyl ester 2 (7.25 g, 62%) as a white solid. ¹H NMR (DMSOH-d₆) δ 11.45 (s, 1H), 9.86 (s, 1 H), 8.20 (s, 1H), 7.38 (m, 1H), 7.26 (m, 2H), 6.03 (s, 1H), 4.15 (t, 2H, J=5.5 Hz), 3.49 (m, 4H), 2.59 (t, 2H, J=5.5 Hz), 2.39 (s, 3H), 2.22 (s, 3H), 1.14 (s, 9H); MS (ESI, M+H⁺) 572, 574.

To a solution of pivalate (2.40 g, 4.2 mmole) in DMF (20 mL) at room temperature was added HCl/dioxane (4N, 1.4 mL). The resultant solution was concentrated under reduced pressure to a light yellow solid, which was triturated with Et₂O to give HCl salt of 2 as a beige colored solid (2.5 g). ¹H NMR (DMSO-d₆) δ 8.14 (s, 1H), 7.33 (m, 1H), 7.23 (m, 2H), 6.08 (s, 1H), 4.28 (m, 1H), 4.16 (m, 5H), 3.32 (m, 6H), 2.40 (s, 3H), 2.17 (s, 3H), 1.11 (s, 9H); MS (ESI, M+H⁺) 572, 574.

### EXAMPLE 2

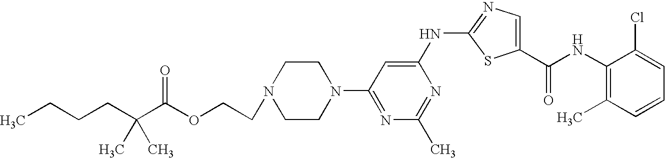

### 2-(4-(6-(5-((2-Chloro-6-methylphenyl)carbamoyl)thiazol-2-ylamino)-2-methylpyrimidin-4-yl)piperazin-1-yl)ethyl 2,2-dimethylhexanoate (3)

To a mixture of 1 (500 mg, 1.02 mM), Et₃N (0.5 mL) and catalytic amount of 4-dimethylaminopyridine in DMF (10 mL) at an ice bath temperature 2,2-dimethylhexanoyl chloride (163 mg, 1 mM, Reference: F. Kienzle and R. E. Minder, Helv. Chim. Acta, 1980, 63, 1425; S. D. Kimball, J. Das, P. Chen, E. I. Iwanowicz, R. E. White, R. Zahler: “Acyl Guanidine and Amidine Prodrugs of Thrombin Inhibitors” European Patent EP-743320-A3, Jun. 7, 2000) in dichloromethane was added slowly. After 30 minutes it was warmed to room temperature and stirred for 6 hours. The reaction does not appear to progress any further. Water (30 mL) was added slowly to the reaction mixture and the precipitate was collected by filtration, which was a mixture of starting material and desired product along with minor impurities. This solid mixture was dissolved in DMF (8 mL) by heating, and then water was added to the solution at room temperature and the precipitated solid was collected by filtration. The same procedure was repeated again with the solid to obtain pure product 3 (65 mg, 10% yield) as a white solid. ¹H NMR (500 MHz, DMSO-d₆) δ 11.49 (s, 1H), 9.90 (s, 1H), 8.24 (s, 1H), 7.30 (d, 1H, J=7.7 Hz), 7.29 (m, 2H), 6.07 (s, 1H), 4.17 (t, 2H, J=5.5 Hz), 3.51 (m, 4H), 2.61 (t, 2H, J=5.5 Hz), 2.52 (m, 4H), 2.42 (s, 3 H), 2.25 (s, 3H), 1.48 (t, 2H, J=8.2 Hz), 1.18˜1.29 (m, 4H), 1.12 (s, 6H), 0.86 (t, 3H, J=7.2 Hz); MS (ESI, M+H⁺) 614, 616.

### EXAMPLE 3

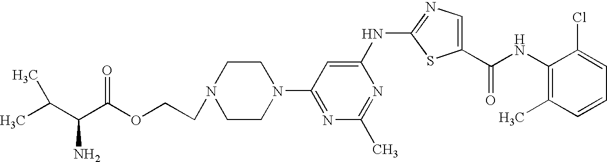

### (S)-2-(4-(6-(5-((2-Chloro-6-methylphenyl)carbamoyl)thiazol-2-ylamino)-2-methyl pyrimidin-4-yl)piperazin-1-yl)ethyl 2-amino-3-methylbutanoate (4)

A. (S)-2-(4-(6-(5-((2-Chloro-6-methylphenyl)carbamoyl)thiazol-2-ylamino)-2-methylpyrimidin-4-yl)piperazin-1-yl)ethyl 2-(tert-butoxycarbonyl)-3-methyl-butanoate. To a stirred mixture of 1 (0.05 g, 0.1 mmole) with EDCI (0.09 g, 0.46 mmole) and dimethylaminopyridine (2 mg, 0.02 mmole) in DMF (1.2 mL) at room temperature under nitrogen was added N-Boc-L-valine (0.07 g, 0.32 mmole). The resultant mixture was stirred at room temperature for 2 hour. Water was added and the precipitated solid was collected which washed successively with water, aqueous NaHCO₃ and Et₂O to obtain (S)-2-(4-(6-(5-((2-chloro-6-methylphenyl)carbamoyl)thiazol-2-ylamino)-2-methylpyrimidin-4-yl)piperazin-1-yl)ethyl 2-(tert-butoxycarbonyl)-3-methyl-butanoate (0.053g, 76%) as a white solid. MS (ESI, M+H⁺) 687, 689.

B. A mixture of (S)-2-(4-(6-(5-((2-chloro-6-methylphenyl)carbamoyl)thiazol-2-ylamino)-2-methylpyrimidin-4-yl)piperazin-1-yl)ethyl 2-(tert-butoxycarbonyl)-3-methyl-butanoate (0.51 g, 0.74 mmole) and trifluoroacetic acid (TFA) (2 mL) in CH₂Cl₂ (2 mL) was stirred at room temperature for 20 min., and all the TFA/CH₂Cl₂ were removed under reduced pressure. The residue was treated with aqueous NaHCO₃ solution adjusting pH to 7-8, and precipitated solid was collected which was washed with H₂O. The solid was further purified by prep HPLC under isocratic condition of preparative HPLC(33 % MeOH in H₂O with 0.1% TFA) to give a TFA salt of 4 as a white solid (12.4 mg).

General preparative HPLC condition: Shimadzu HPLC machine was used with a YMC PAK ODS, 100×20 mm, S-5μ column eluting with a mixture of solvent A and B (starting from 30% solvent B and 70% Solvent A to 100% B, gradient over 10 minutes); Solvent A: 90% H₂O-10% MeOH-0.1% TFA, Solvent B: 90% MeOH-10% H₂O-0.1% TFA.

¹H NMR (DMSO-d₆) δ 9.86 (s, 1H), 8.21 (s, 1H), 7.39 (d, 1H,. J=6.6 Hz)), 7.26 (m, 2H), 6.04 (s, 1H), 4.23 (m, 1H), 4.13 (m, 2H), 3.50 (s, 4H), 3.15 (d, 1 H, J=4.95 Hz), 3.11 (d, 1H, J=5.45 Hz), 2.58 (m, 2H), 2.39 (s, 3H), 2.23 (s, 3H), 0.88 (d, 3H, J=6.6 Hz), 0.83 (d, 3H, J=6.6 Hz); ¹³C NMR (DMSO-d₆) δ 174.9, 164.8, 162.1, 161.9, 156.6, 140.5, 138.2, 133.0, 132.0, 128.6, 128.0, 126.7, 125.5, 82.3, 60.8, 59.1, 55.7, 52.0, 43.3, 31.6, 25.2, 18.8, 18.0, 17.1; MS (ESI, M+H⁺) 587, 589.

### EXAMPLE 4

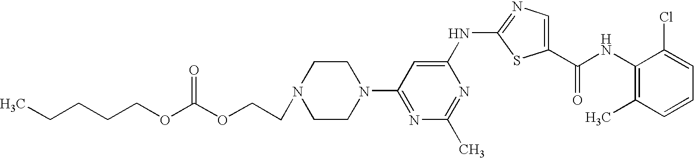

### 2-(4-(6-(5-((2-chloro-6-methylphenyl)carbamoyl)thiazol-2-ylamino)-2-methyl pyrimidin-4-yl)piperazin-1-yl)ethyl pentyl carbonate (5)

To a stirred mixture of 1 (0.50 g, 1.02 mmole), Et₃N (0.43 mL, 3.09 mmole) and dimethylaminopyridine (12 mg, 0.1 mmole) in DMF (5 mL) at room temperature under nitrogen was added n-amyl chloroformate (0.47 mL, 3.08 mmole). The resultant mixture was stirred at room temperature for 6 hour. It was added with water and precipitated solid was collected which washed with water and Et₂O. The crude solid was re-crystallized twice from DMF-H₂O to obtain 5 (0.21 g, 34%) as a white solid. ¹H NMR (DMSO-d₆) δ 11.47 (s, 1H),9.87 (s, 1H), 8.20 (s, 1H), 7.38 (d, 1H, J=7.7 Hz), 7.26 (m, 2H), 6.04 (s, 1 H), 4.19 (t, 2H, J=5.5 Hz), 4.05 (t, 2H, J=6.88 Hz), 3.49 (m, 4H), 2.59 (t, 2H, J=5.5 Hz), 2.40 (s, 3H), 2.23 (s, 3H), 1.58 (t, 2H, J=6.88 Hz), 1.28 (m, 4H), 0.85 (t, 3 H, J=6.88 Hz); ¹³C NMR (DMSO-d₆) δ 164.9, 162.2, 161.9, 159.5, 156.6, 154.3, 140.4, 138.5, 133.3, 132.2, 128.8, 128.02, 126.6, 125.5, 67.2, 64.1, 55.76, 52.0, 43.3, 27.5, 27.0, 25.3, 21.4, 18.0, 13.5; MS (ESI, M+H⁺) 602, 604.

### EXAMPLE 5

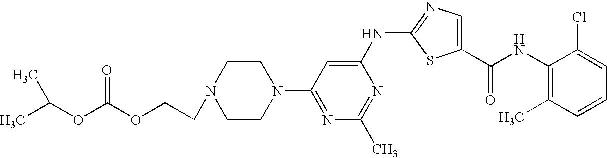

### 2-(4-(6-(5-((2-Chloro-6-methylphenyl)carbamoyl)thiazol-2-ylamino)-2-methylpyrimidin-4-yl)piperazin-1-yl)ethyl isopropyl carbonate (6)

To a mixture of 1 (488 mg, 1 mM), Et₃N (3 mL) and 4-dimethylaminopyridine (50 mg) in DMF (10 mL) at room temperature isopropyl chloroformate solution (3.5 mL of 1 M solution in toluene, 3.5 mM) was added slowly. After 4.5 hour water (30 mL) and EtOAc (70 mL) were added and the organic layer was separated. The organic solution was dried over MgSO₄, concentrated and the product was purified by preparative HPLC to obtain TFA salt of 6 (68 mg, 12% yield) as a white solid. ¹H NMR (500 MHz, MeOD-d₃) δ 8.08 (s, 1H), 7.25 (d, 1H, J=7.7 Hz), 7.15 (m, 2H), 6.11 (s, 1H), 4.41 (t, 2H, J=5.5 Hz), 3.38˜4.00 (m, 11H), 2.44 (s, 3H), 2.22 (s, 3 H), 1.20 (d, 6H, J=6.6 Hz); MS (ESI, M+H⁺) 574, 576.

### EXAMPLE 6

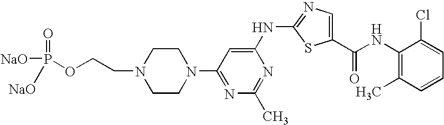

### Sodium 2-(4-(6-(5-((2-chloro-6-methylphenyl)carbamoyl)thiazol-2-ylamino)-2-methylpyrimidin-4-yl)piperazin-1-yl)ethyl phosphate (9)

A. Preparation of phosphate 8: To a solution of 1 (487 mg, 1 mM) in DMF (5 mL) at room temperature di-tert-butyl dimethylphosphoramidite (0.280 mL, 1 mM) was added followed by tetrazole (135 mg), and the reaction mixture was stirred for 30 min. Additional amount of di-tert-butyl dimethylphosphoramidite (0.55 mL) and tetrazole (190 mg) were added. The reaction was complete cleanly in 30 min. to afford phosphite 7. Next step of oxidation reaction was carried out in situ without any purification of the phosphite intermediate 7. It was cooled to −78° C. and was added ˜60% mCPBA (650 mg) and stirred for 40 min. DMSO (0.5 mL) was added to the mixture at −70° C. and the mixture was stirred for 15 min. Aqueous NaHCO₃ (20 mL) and EtOAc (70 mL) were added to the mixture and the organic layer was separated, dried over MgSO₄, concentrated and the residue was triturated with ether to obtain the crude product of phosphate 8 (485 mg, 71% overall yield) as a white solid. This product contained a minor impurity peak according to the HPLC analysis at 220 nm, but its ¹H NMR looked very good. Thus, it was used directly for the next step without any further purification.

¹H NMR (500 MHz, MeOD-d₃) δ 8.15 (s, 1H), 7.35 (d, 1H, J=7.7 Hz), 7.22(m, 2 H), 6.00 (s, 1H), 4.13 (t, 2H, J=5.5 Hz), 3.68 (m, 4H), 2.74 (t, 2H, J=5.5 Hz), 2.64 (m, 4H), 2.47 (s, 3H), 2.32 (s, 3H), 1.50 (s, 18H).

B. Preparation of 9. To the heterogeneous mixture of phosphate obtained above (485 mg, 0.71 mM) in CH₂Cl₂ at an ice bath temperature was added TFA (3 mL) slowly. Upon addition of TFA it soon became a homogeneous solution. After 30 min. it was warmed to room temperature and allowed to stay for 2 hour. It was concentrated and the residue was partially purified by preparative HPLC. The partially purified product was dissolved in 1 N NaOH (10 mL) and methanol (10 mL), mostly concentrated and the remaining material was passed through a HP-20 resin column eluting with water followed by 30% MeOH in water, 50% MeOH in water to obtain phosphate bis-sodium salt 9 (250 mg, 58% yield) as a white solid after lyophilization. ¹H NMR (500 MHz, MeOD-d₃) δ 8.14 (s, 1H), 7.35 (d, 1H, J=7.7 Hz), 7.24 (m, 2H), 6.01 (s, 1H), 4.00 (t, 2H, J=5.5 Hz), 3.68 (m, 4H), 2.70 (t, 2H, J=5.5 Hz), 2.67 (m, 4H), 2.47 (s, 3H), 2.32 (s, 3H); MS (ESI, M+H⁺) 568, 570.

### EXAMPLE 7

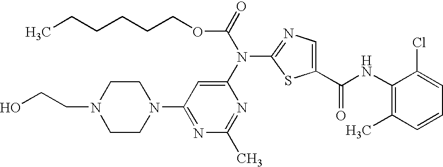

### Hexyl 5-((2-chloro-6-methylphenyl)carbamoyl)thiazol-2-yl(6-(4-(2-hydroxyethyl)piperazin-1-yl)-2-methylpyrimidin-4-yl)carbamate (11)

A. Silylation for the preparation of 2-(6-(4-(2-(tert-butyldimethylsilyloxy)ethyl)piperazin-1-yl)-2-methylpyrimidin-4-ylamino)-N-(2-chloro-6-methylphenyl)thiazole-5-carboxamide (10). To a stirred mixture of 1 (5.0 g, 10.2 mmole) in DMF (60 mL) with Et₃N (4.3 mL, 30.9 mmole) at room temperature under nitrogen was added t-butyldimethylsilyl chloride (5.0 g, 33.2 mmole) and dimethylaminopyridine (0.06g, 0.5 mmole). The resultant mixture was stirred at room temperature for 1.5 hour. It was added with water and precipitated solid was collected which washed with water, and dried in vacuo at 35-40° C. over MgSO₄ overnight to give 10 (5.70 g, 93.4%) as a white solid. ¹H NMR (DMSO-d₆) δ 9.91 (s, 1H), 8.24 (s, 1H), 7.40 (d, 1H, J=7.15 Hz), 7.26 (m, 2H), 6.09 (s, 1H), 3.73 (m, 2H), 3.48 (m, 4H), 3.33 (m, 2H), 2.50 (m, 2H), 2.37 (s, 3H), 2.24 (s, 3H), 0.87 (s, 6H), 0.07 (s, 9H)); MS (ESI, M+H⁺) 602, 604

B. Coupling Reaction. To a stirred mixture 10 (0.40 g, 0.64 mmole) in anhydrous pyridine (24 mL) with DMAP (8 mg, 0.07 mmole) at room temperature under nitrogen was added hexyl chloroformate (0.33 mL, 1.0 mmole). The resultant mixture was stirred at room temperature and for every 20-30 min, additional hexyl chloroforamte (0.22 mL) was added to keep the reaction proceeding until the reaction no longer progress. The reaction mixture was quenched with aqueous NaHCO₃ solution, extracted with EtOAc (3×30 mL) and dried over MgSO₄. It was concentrated and treaturated with CH₂Cl₂. The unreacted starting material 10 was precipitated out as a white solid. After filtering the solid the filtrate solution was concentrated to an oil and this crude product was used in the next step without any further purification. MS (ESI, M+H⁺) 616, 618.

C. Desilylation Reaction. To a stirred solution of the product obtained above in THF (12 mL) at room temperature under nitrogen was added a solution of tetra-n-butylammonium fluoride (1.0 M in THF, 2.0 mL). After 50 min., the reaction mixture was quenched with HOAc (1 mL), then diluted with EtOAc to 70 mL, organic layer was separated, washed with water, aqueous NaHCO₃ solution and then by brine. The EtOAc solution was dried over MgSO₄ and concentrated to a brown oil. Purification by prep HPLC gave a TFA salt of 11 as a light yellow solid (0.115 g, 46%). ¹H NMR (CDCl₃) δ 7.66 (s, 1H), 7.28 (d, 1H, J=7.7 Hz)), 7.22 (t, 1H, J=7.7 Hz), 7.14 (m, 2 H), 4.12 (m, 2H), 4.05 (m, 4H), 3.95 (s, 2H), 3.30 (m, 4H), 3.12 (s, 2H), 2.44 (s, 3 H), 2.18 (s, 3H), 1.47 (m, 2H), 1.10 (m, 6H), 0.75 (t, 3H, J=6.88 Hz);; ¹³C NMR (CDCl₃) δ 162.2, 161.5, 152.8, 138.8, 134.6, 133.5, 130.2, 129.6, 127.9, 117.3, 115.8, 84.5, 68.0, 60.0, 56.2, 52.5, 41.3, 31.1, 28.3, 25.2, 22.4, 18.3, 13.9; MS (ESI, M+H⁺) 616, 618.

### EXAMPLE 8

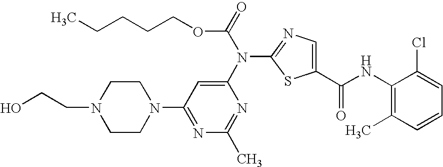

### Pentyl 5-((2-chloro-6-methylphenyl)carbamoyl)thiazol-2-yl(6-(4-(2-hydroxyethyl)piperazin-1-yl)-2-methylpyrimidin-4-yl)carbamate (12)

Analogue 12 was prepared in the same way as compound 11 using n-pentyl chloroformate. ¹H NMR (500 MHz, MeOD-d₃) δ 7.83 (s, 1H), 7.41 (d, 1H, J=7.7 Hz), 7.35 (t, 1H, J=7.7 Hz), 7.31 (d, 1H, J=7.7 Hz), 6.12 (s, 1H), 4.20 (m, 2H), 3.91 (t, 2H, J=5.5 Hz), 3.30 (m, 9H), 2.47 (s, 3H), 2.27 (s, 3H), 1.15˜1.50 (m, 7H), 0.82 (t, 3H, J=7.2 Hz); MS (ESI, M+H⁺) 602, 604.

### EXAMPLE 9

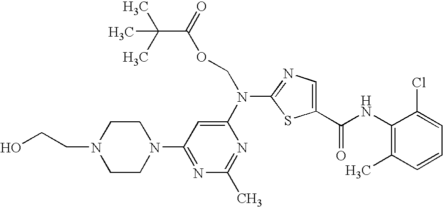

### ((5-((2-chloro-6-methylphenyl)carbamoyl)thiazol-2-yl)(6-(4-(2-hydroxyethyl)piperazin-1-yl)-2-methylpyrimidin-4-yl)amino)methyl pivalate (13)

A. Coupling Reaction. To a stirred mixture 10 (0.20 g, 0.33 mmole) in anhydrous DMF (4 mL) with Cs₂CO₃ (0.25 g, 0.77 mmole) at room temperature under nitrogen was added chloromethyl pivalate (0.06 mL, 0.42 mmole, prepared according to the procedure described in P. Gomes, M. I. Santos, M. J. Trigo, R. Castanheiro, and R. Moreira, Syn. Comm., 2003, 33, 1683). The resultant mixture was stirred at room temperature overnight. The reaction mixture was added with H₂O and was extracted with EtOAc (3×30 mL). The EtOAc solution was washed with 10% LiCl solution twice, dried over MgSO₄, and concentrated in vacuo. Purification by prep HPLC provided a partially purified product of coupled silyl compound as a white solid which was used directly in the next step. MS (ESI, M+H⁺) 716, 718.

B. Desilylation Reaction. To a stirred solution of the product obtained above in THF (5 mL) at room temperature under nitrogen was added a solution of tetra-n-butylammonium fluoride (1.0 M in THF, 1.7 mL). After 3 hour it was quenched with HOAc (0.8 mL), then diluted with EtOAc to 80 mL. The organic layer was taken, washed with brine twice, dried over MgSO₄ and concentrated to a brown oil. Purification by prep HPLC gave a TFA salt of 13 (0.04 g, 27%) as a white solid. ¹H NMR (DMSO-d₆) δ 9.96 (s, 1H), 9.75 (s, 1H), 8.23 (s, 1H), 7.34 (d, 1H, J=7.7 Hz), 7.22 (m, 2H), 6.45 (s, 1H), 6.32 (s, 2H), 4.52 (m, 2H), 3.70 (m, 2H), 3.53 (m, 2H), 3.29 (m, 2H), 3.16 (m, 2H), 3.05 (m, 2H), 2.43 (s, 3H), 2.17 (s, 3H), 1.05 (m, 9H); MS (ESI, M+H⁺) 602, 604.

### EXAMPLE 10

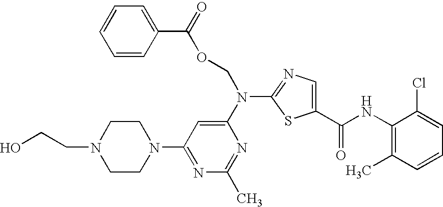

### ((5-((2-chloro-6-methylphenyl)carbamoyl)thiazol-2-yl)(6-(4-(2-hydroxyethyl)pipera-zin-1-yl)-2-methylpyrimidin-4-yl)amino)methyl benzoate (14)

A. Coupling Reaction. To a stirred mixture of with NaH (60%, 50 mg, 1.3 mmole) in anhydrous DMF (6 mL) at room temperature under nitrogen was added 2-(6-(4-(2-(tert-butyl-dimethylsilyloxy)ethyl)piperazin-1-yl)-2-methylpyrimidin-4-ylamino)-N-(2-chloro-6-methylphenyl)thiazole-5-carboxamide, 10 (0.20 g, 0.34 mmole). The resultant mixture was stirred at room temperature for 8 min., then treated with chloromethyl benzoate (0.2 g, 1.2 mmole), the reaction mixture was stirred at room temperature overnight. The reaction mixture was added with aqueous NH₄Cl solution and extracted with EtOAc (3×45 mL). The EtOAc solution was washed with 10% LiCl solution and concentrated to an oily residue. The residue was purified by prep. HPLC to give a white solid as impure product and used directly in next step. MS (ESI, M+H⁺) 736, 738.

B. Desilylation Reaction. To a stirred solution of the product obtained above in THF (4 mL) at room temperature under nitrogen was added a solution of tetra-n-butylammonium fluoride (1.0 M in THF, 1.5 mL), the reaction mixture was stirred at room temperature overnight. It was quenched with HOAc (1 mL), and concentrated to a brown oil. Purification of the crude product by prep HPLC gave a TFA salt of 14 (0.055 g, 36%) as a white solid. ¹H NMR (DMSO-d₆) δ 9.96 (s, 1H), 9.66 (s, 1H), 8.23 (s, 1H), 7.86 (d, 1H, J=7.7 Hz), 7.61 (t, 1H, J=7.42 Hz), 7.45 (t, 2H, J=7.7 Hz),7.33 (d, 1H, J=7.7 Hz), 7.21 (m, 2H), 6.64 (s, 1H), 6.58 (s, 2H), 5.36 (s, 1H), 4.54 (m, 2H), 3.68 (m, 2H), 3.49 (m, 2H), 3.30 (m, 2H), 3.14 (m, 2H), 3.10 (m, 2 H), 2.53 (s, 3H), 2.22 (s, 3H); MS (ESI, M+H⁺) 622, 624.

### EXAMPLE 11

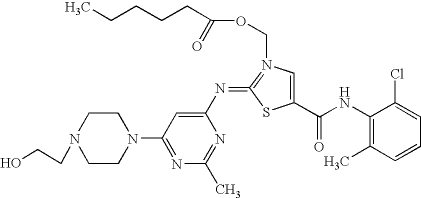

### ((5-((2-chloro-6-methylphenyl)carbamoyl)thiazol-2-yl)(6-(4-(2-hydroxyethyl)pipera-zin-1-yl)-2-methylpyrimidin-4-yl)amino)methyl hexanoate (15)

A. Coupling Reaction. To a stirred mixture of with NaH (60%, 40 mg, 1.0 mmole) in anhydrous DMF (5 mL)) at room temperature under nitrogen was added 10 (0.20 g, 0.33 mmole). The resultant mixture was stirred at room temperature for 25 min., then was added with chloromethyl hexanoate (0.3 g, 1.8 mmole, prepared according to the procedure described in P. Gomes, M. I. Santos, M. J. Trigo, R. Castanheiro, and R. Moreira, Syn. Comm., 2003, 33, 1683). The reaction mixture was stirred at room temperature overnight. The reaction mixture were added with additional NaH (20 mg, 0.5 mmole) and chloromethyl hexanoate (0.15 g, 0.9 mmole) and the reaction mixture was stirred at room temperature for 3 hour. It was added with aqueous NH₄Cl solution and extracted with EtOAc (3×25 mL). The EtOAc solution was washed with 10% LiCl solution twice and concentrated. Purification of the crude product by prep. HPLC gave a partially purified coupled silyl product (0.1 g) as a white solid and it was used directly in the next step. MS (ESI, M+H⁺) 730, 732.

B. Desilylation Reaction. To a stirred solution of the above solid in THF (4 mL) at room temperature under nitrogen was added a solution of tetra-n-butylammonium fluoride (1.0 M in THF, 1.0 mL), the reaction mixture was stirred at room temperature for 1.5 hour. It was quenched with HOAc (0.5 mL) and the mixture was directly purified by prep HPLC to give a TFA salt of 15 (0.052 g, 52%) as a white solid. ¹H NMR (DMSO-d₆) δ 10.8 (s, 1H), 9.77 (s, 1H), 8.33 (s, 1H), 7.40 (m, 1H), 7.28 (m, 2H), 6.36 (s, 1H), 6.06 (s, 2H), 4.52 (m, 2H), 3.76 (m, 2H), 3.55 (m, 2H), 3.30 (m, 2H), 3.23 (m, 2H), 3.09 (m, 2H), 2.46 (s, 3H), 2.39 (t, 2H, J=7.15 Hz), 2.22 (s, 3 H), 1.55 (m, 2H), 1.24 (m, 4H), 0.82 (t, 3H, J=6.88 Hz); ¹³C NMR (DMSO-d₆) δ 172.6, 164.0, 162.6, 158.6, 158.0, 157.7, 138.4, 132.9, 132.0, 128.9, 128.1, 126.9, 116.7, 68.4, 57.3, 54.5, 50.5, 40.5, 32.9, 30.3, 23.7, 21.5, 19.0, 13.5; MS (ESI, M+H⁺) 616, 618.

### EXAMPLES 12 AND 13

### ((5-((2-chloro-6-methylphenyl)carbamoyl)thiazol-2-yl)(6-(4-(2-hydroxyethyl)piperazin-1-yl)-2-methylpyrimidin-4-yl)amino)methyl 2,2-dimethylhexanoate (16)

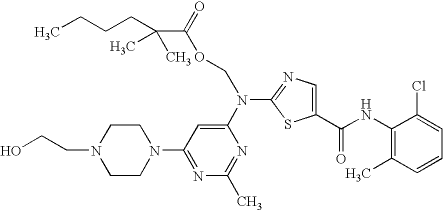

### and (Z)-(5-((2-chloro-6-methylphenyl)carbamoyl)-2-(6-(4-(2-yl)methyl 2,2-dimethylhexanoate (17)

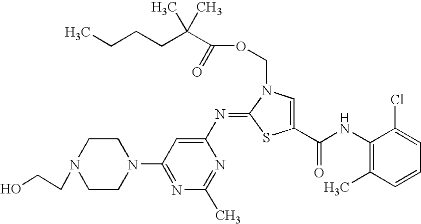

A. Preparation of Chloromethyl α,α-dimethylhexanoate. A mixture of αα-dimethylhexanoic acid (0.91 g, 6.31 mmole) in DMF (10 mL) and Cs₂CO₃ (2.70 g, 8.3 mmole) was stirred for 20 min. at room temperature under nitrogen, and was added with bromochloromethane (5 mL, 77 mmole). The resultant mixture was stirred at room temperature overnight. The reaction mixture was filtered through a layer of MgSO4 and the filtrate solution was concentrated to 10 mL in volume to give chloromethyl α,α-dimethylhexanoate in DMF (1.2 g/10 mL) which was used directly in the next step (Reference: P. Gomes, M. I. Santos, M. J. Trigo, R. Castanheiro, and R. Moreira, Syn. Comm., 2003, 33, 1683).

B. Coupling Reaction. To a stirred mixture of with NaH (60%, 90 mg, 2.3 mmole) in anhydrous DMF (6 mL)) at room temperature under nitrogen was added 10 (0.60 g, 1.0 mmole). The resultant mixture was stirred at room temperature for 20 min., then treated with chloromethyl α,α-dimethylhexanoate solution obtained above (0.58 g, 3.0 mmole). The reaction mixture was stirred at room temperature overnight. The reaction mixture was added with aqueous NH₄Cl solution, extracted with EtOAc (3×60 mL). The EtOAc solution was separated, washed with 10% LiCl solution and concentrated to an oil which was purified by prep. HPLC to give a TFA salts of 16 and 17 as white solids.

tert-Butyldimethylsilyl ether of 16 TFA salt: 0.15 g, 17.2%, ¹H NMR (CDCl₃) δ 7.41 (m, 1H)), 7.31 (m, 1H), 7.19 (m, 2H), 6.38 (s, 2H), 6.21 (s, 1H), 4.51 (m, 2 H), 4.04 (m, 2H), 3.80 (m, 2H), 3.55 (m, 2H), 3.24 (m, 2H), 2.96 (m, 2H), 2.56 (m, 3H), 2.33 (m, 3H), 1.47 (m, 2H), 1.15 (m, 8H), 0.88 (m, 8H), 0.77 (t, 3H, J=7.13 Hz), 0.08 (s, 9H); MS (ESI, M+H⁺) 758, 760.

tert-Butyldimethylsilyl ether of 17 TFA salt: 0.093 g, 10.7%, ¹H NMR (CDCl₃) δ 7.99 (s, 1H), 7.20 (m, 1H)), 7.09 (m, 2H), 6.11 (s, 1H), 5.91 (s, 2H), 3.90 (m, 3 H), 3.13 (m, 3H), 2.50 (m, 4H), 2.23 (m, 4H), 1.40 (m, 2H), 1.07 (m, 6H), 0.98 (m, 2H), 0.79 (s, 6H), 0.71 (t, 3H, J=7.42 Hz), 0.00 (s, 9H); MS (ESI, M+H⁺) 758, 760.

C. DesilylationReaction. To a stirred solution of ((5-((2-chloro-6-methylphenyl)carbamoyl)thiazol-2-yl)(6-(4-(2-(tert-butyldimethylsilyloxy)ethyl)piperazin-1-yl)-2-methylpyrimidin-4-yl)amino)methyl 2,2-dimethylhexanoate (0.15 g, 0.17 mmole)in THF (6 mL) at room temperature under nitrogen was added tetra n-butylammonium fluoride (1.0 M in THF, 2.0 mL) and the reaction mixture was stirred at room temperature for 3 hour. It was quenched with HOAc (1.0 mL), and concentrated to a brown oil which was purified by prep HPLC to give a TFA salt of 16 (0.1265 g, 97%) as a white solid. ¹HNMR (DMSO-d₆) δ 10.03 (s, 1H), 8.31 (s, 1H), 7.40 (d, 1H, J=7.7 Hz)), 7.28 (m, 2H), 6.55 (s, 1 H), 6.41 (s, 2H), 4.58 (m, 2H), 3.77 (m, 2H), 3.529 (m, 2H), 3.40 (m, 2H), 3.24 (m, 2H), 3.11 (m, 2H), 2.50 (s, 3H), 2.24 (s, 3H), 1.38 (m, 2H), 1.07 (m, 10H), 0.75 (t, 3H, J=6.318 Hz)); MS (ESI, M+H⁺), 644, 646.

To a stirred solution of (Z)-(2-(6-(4-(2-(tert-butyldimethylsilyloxy)ethyl)piperazin-1-yl)-2-methylpyrimidin-4-ylimino)-5-((2-chloro-6-methylphenyl)carbamoyl)thiazol-3(2H)-yl)methyl 2,2-dimethyl-hexanoate (0.093 g, 0.1 mmole) in THF (4 mL) at room temperature under nitrogen was added tetratbutylammonium fluoride (1.0 M in THF, 1.0 mL) and the reaction mixture was stirred at room temperature for 3.5 hour. It was quenched with HOAc (1.2 mL), and concentrated to a brown oil which was purified by prep HPLC to give a TFA salt of 17 (0.076 g, 88%) as a white solid. ¹H NMR (DMSO-d₆) δ 10.06 (s, 1H), 8.32 (s, 1H), 7.40 (d, 1H, J=7.7 Hz)), 7.30 (m, 2 H), 6.34 (s, 1H), 6.08 (s, 2H), 4.51 (m, 2H),3.55 (m, 2H), 3.29 (m, 2H), 3.23 (m, 2 H), 3.09 (m, 2H), 2.46 (s, 3H), 2.22 (s, 3H), 1.44 (m, 2H), 1.15 (s, 6H), 1.13 (m, 2 H), 1.08 (m, 2H), 0.71 (t, 3H, J=6.88 Hz); MS (ESI, M+H⁺) 644, 646.

### EXAMPLES 14

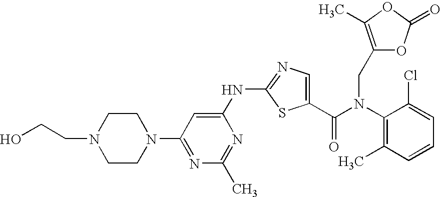

### N-(2-chloro-6-methylphenyl)-2-(6-(4-(2-hydroxyethyl)piperazin-1-yl)-2-methylpyrimidin-4-ylamino)-N-((5-methyl-2-oxo-1,3-dioxol-4-yl)methyl)thiazole-5-carboxamide (19)

A mixture of 10 (0.20 g, 0.33 mmole), 4-(bromomethyl)-5-methyl-1,3-dioxol-2-one (0.16 g, 0.83 mmole) and Cs₂CO₃ (0.23 g, 0.71 mmole) in DMF (9 mL) was stirred overnight at room temperature under nitrogen. The reaction mixture was added with water, the solid was filtered and washed with water and dried to give 18 as a beige solid (0.21 g, 89%).

To a stirred solution of 18 (0.21 g, 0.29 mmole) in THF (8 mL) at room temperature under nitrogen was added tetratbutylammonium fluoride (1.0 M in THF, 2.0 mL), the reaction mixture was stirred at room temperature for 35 min. It was quenched with HOAc (1.0 mL), and concentrated to a brown oil. Purification by prep HPLC gave 19 (68 mg, 32%) as a brown solid. ¹H NMR (DMSO-d₆) δ 11.45 (s, 1H), 7.47 (m, 1H), 7.40 (m, 2H)), 7.30 (s, 1H), 5.98 (s, 1H), 4.81 (d, 1H, J=15.93 Hz), 4.53 (d, 1H, J=15.93 Hz), 4.21 (m, 2H), 3.68 (m, 2H), 3.49 (m, 2H), 3.19 (m, 4H), 3.00 (m, 2H), 2.25 (s, 3H), 2.17 (s, 3H), 1.86 (s, 3H); MS (ESI, M⁺+H), 600, 602.

What is claimed is:

1. A compound of formula (Ia), (Ib), and/or (Ic) or a salt thereof: 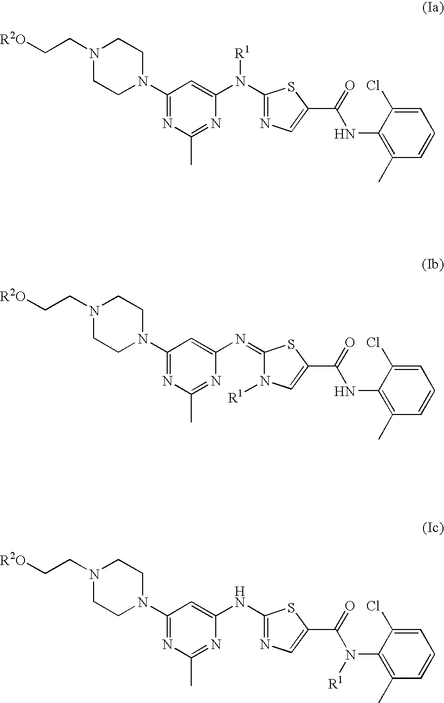
or stereoisomer or pharmaceutically acceptable salt form thererof, wherein
R¹ is selected from H, —P(O)(ONa)₂, —C(R^{a})₂—R³, —C(O)—R, —C(O)O—R;
R² is selected from H, —P(O)(ONa)₂, —C(R^{a})₂—R³, —C(O)—R, —C(O)O—R, —C(O)N(R^{a})₂;
with the provisio that R¹ and R² do not both equal H;
R is independently selected from alkyl, haloalkyl, alkenyl, alkynyl, aryl, heteroaryl, cycloalkyl, carbocyclyl, heterocyclyl, each optionally substituted with one or more Z₁;
R³ is selected from NHC(O)R^{a}, OC(O)R^{a}, —OP(O)(ONa)₂, NHC(O)OR, OC(O)OR, OC(O)NHR, 5-methyl-2-oxo-[1,3]dioxol-4-yl, and alkyl 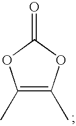
Z¹is selected from alkyl, haloalkyl, alkenyl, alkynyl, aryl, heteroaryl, cycloalkyl, carbocyclyl, heterocyclyl, NR^{a}R^{a}, OR^{a}, NC(O)R^{a}, OC(O)R^{a}; and
R^{a} is independently selected from hydrogen, alkyl, haloalkyl, alkenyl, alkynyl, aryl, heteroaryl, cycloalkyl, carbocyclyl, heterocyclyl.

2. A compound of claim 1 wherein the compound is of formula (Ia) and
R¹ is H; and
R² is selected from —P(O)(ONa)₂, —C(R^{a})₂—R³, —C(O)—R, —C(O)O—R, —C(O)N(R^{a})₂.

3. A compound of claim 1 wherein
R² is selected from —P(O)(ONa)₂, —C(O)—R, —C(O)O—R; and
R is independently selected from alkyl, haloalkyl, aryl, heteroaryl, cycloalkyl, heterocyclyl, each optionally substituted with one or more Z₁.

4. A compound of claim 1 wherein
R is independently selected from alkyl, haloalkyl, each optionally substituted with one or more Z₁;
Z₁is independently selected from alkyl and NR^{a}R^{a}; and
R^{a} is independently selected from hydrogen, alkyl.

5. A compound of claims 4 wherein
R is independently selected from methyl, ethyl, propyl, i-propyl, butyl, s-butyl, i-butyl, t-butyl, pentyl, hexyl, each optionally substituted with one or more Z₁.

6. A compound of claim 1, wherein
R¹ is selected from —P(O)(ONa)₂, —C(R^{a})₂—R³, —C(O)—R, —C(O)O—R;
R² is selected from H.

7. A compound of claim 6, wherein
R¹ is selected from —P(O)(ONa)₂, —C(R^{a})₂—R³, —C(O)—R, —C(O)O—R;
R is independently selected from alkyl, haloalkyl, aryl, heteroaryl, cycloalkyl, heterocyclyl, each optionally substituted with one or more Z₁;
R³ is selected from NHC(O)R^{a}, OC(O)R^{a}, —OP(O)(ONa)₂, NHC(O)OR, OC(O)OR, OC(O)NHR, and alkyl

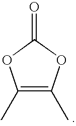

8. A compound of claims 7, wherein
R is independently selected from alkyl, haloalkyl, each optionally substituted with one or more Z₁;
Z₁is independently selected from alkyl and NR^{a}R^{a}; and
R^{a} is independently selected from hydrogen, alkyl.

9. A compound of claims 8, wherein
R¹ is selected from —C(R^{a})₂—R³, —C(O)O—R;
R is independently selected from methyl, ethyl, propyl, i-propyl, butyl, s-butyl, i-butyl, t-butyl, pentyl, hexyl, each optionally substituted with one or more Z₁;
R³ is OC(O)R^{a}, and alkyl

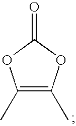

10. A compound of claims 9 wherein the compound is a compound of formula (Ia).

11. A compound of claims 9 wherein the compound is a compound of formula (Ib).

12. A compound of claims 9 wherein the compound is a compound of formula (Ic).

13. A pharmaceutical composition, comprising a pharmaceutically acceptable carrier and a therapeutically effective amount of a compound of claims 1.

14. The method for the treatment of a protein tyrosine kinase-associated disorder, comprising the step of administering to a subject in need thereof an amount effective therefor of at least one compound claims 1 or a salt thereof, wherein said protein tyrosine kinase-associated disorder is selected from transplant rejection, rheumatoid arthritis, multiple sclerosis, lupus, graft vs. host disease, a T-cell mediated hypersensitivity disease, psoriasis, Hashimoto's thyroiditis, Guillain-Barre syndrome, cancer, contact dermatitis, an allergic disease, asthma ischemic or reperfusion injury, atopic dermatitis, allergic rhinitis, chronic obstructive pulmonary disease, diabetic retinopathy.

15. The method of claim 14, wherein said protein tyrosine-kinase-associated disorder is cancer.

16. The method of claim 15 wherein the cancer is chronic myelogenous leukemia (CML), gastrointestinal stromal tumor (GIST), acute lymphoblastic leukemia (ALL), small cell lung cancer (SCLC), non-small cell lung cancer (NSCLC), ovarian cancer, melanoma, mastocytosis, germ cell tumors, acute myelogenous leukemia (AML), pediatric sarcomas, breast cancer, colorectal cancer, pancreatic cancer, or prostate cancer.
